(19) United States
(12) Patent Application Publication
(10) Pub. No.: US 2024/0229139 A9
(43) Pub. Date: Jul. 11, 2024

(54) METHODS OF PREDICTING MULTISYSTEM INFLAMMATORY SYNDROME (MIS-C) WITH SEVERE MYOCARDITIS IN SUBJECTS SUFFERING FROM A SARS-COV2 INFECTION OR DISEASE SEVERITY FOLLOWING SARS-COV-2 INFECTION OR MYOCARDITIS POST-VACCINATION AGAINST SARS-COV-2

(71) Applicants: INSERM (INSTITUT NATIONAL DE LA SANTÉ ET DE LA RECHERCHE MÉDICALE, Paris (FR); UNIVERSITÉ PARIS CITÉ, Paris (FR); FONDATION IMAGINE, Paris (FR); ASSISTANCE PUBLIQUE-HÔPITAUX DE PARIS (APHP), Paris (FR)

(72) Inventors: Mickaël MENAGER, Paris (FR); Julie TOUBIANA, Paris (FR); Darragh DUFFY, Paris (FR); Frédéric LAUCAT, Paris (FR)

(21) Appl. No.: 18/546,611

(22) PCT Filed: Feb. 16, 2022
(86) PCT No.: PCT/EP2022/053839

§ 371 Date: Aug. 16, 2023

(30) Foreign Application Priority Data
Feb. 17, 2021 (EP) 21305197.2

(51) Int. Cl. C12Q 1/6883

(52) U.S. Cl. CPC C12Q 1/6883; C12Q 2600/118; C12Q 2600/158

(57) ABSTRACT

SARS-CoV-2 infection in children is generally milder than in adults, yet a proportion of cases result in hyperinflammatory conditions often including myocarditis. To better understand these cases, the inventors applied a multi-parametric approach to the study of blood cells of 56 children hospitalized with suspicion of SARS-CoV-2 infection. The most severe forms of MIS-C (multisystem inflammatory syndrome in children related to SARS-CoV-2), that resulted in myocarditis, were characterized by elevated levels of pro-angiogenesis cytokines and several chemokines. This phenotype was associated with TNF-α signaling, sustained NF-κB signaling in monocytic/dendritic cells, alongside increased HIF-1α and VEGF signaling. Single-cell transcriptomic analyses identified

### FIELD OF THE INVENTION

The present invention is in the field of medicine, in particular virology and inflammation.

### BACKGROUND OF THE INVENTION

In adults, severe forms of COVID-19 are typically characterized by severe pneumonia and acute respiratory distress syndrome (Wiersinga et al., 2020). In children, symptomatic COVID-19 occurs much less frequently and is milder than in adults, with multifactorial reasons for these differences (Brodin, 2020; Castagnoli et al., 2020; Gudbjartsson et al., 2020; Levy et al., 2020; Tagarro et al., 2020). However, in regions with high incidence of SARS-CoV-2 infection, some children have presented a postacute hyperinflammatory illness (Datta et al., 2020). In these cases, diagnostic evidence of recent SARS-CoV-2 infection has been consistently reported (Abrams et al., 2020; Jones et al., 2020; Toubiana et al., 2020, 2021). This condition was named multisystem inflammatory syndrome (MIS-C) or alternatively PIMS-TS (Pediatric Inflammatory Multisystem Syndrome Temporally Associated with SARS-CoV-2)(Whittaker et al., 2020). MIS-C cases most often presented with symptoms similar to Kawasaki disease (KD), an hyperinflammatory illness characterized by clinical features such as strawberry-like tongue and red and dry lips, bulbar conjunctival injection, cervical lymphadenopathy, swollen extremities and diffuse rash (McCrindle et al., 2017). KD complications can develop as myocarditis or shock syndrome in a minority of cases (Kanegaye et al., 2009). KD is thought to be triggered by viral or bacterial pathogens but the precise pathophysiological mechanisms remain elusive, with one hypothesis proposing a superantigen-driven uncontrolled inflammatory immune response (Chang et al., 2014). Compared to classic KD, MIS-C occurs in patients who are older, have more often gastrointestinal symptoms, myocarditis and shock syndrome, and exhibit higher levels of inflammatory markers (Abrams et al., 2020; Datta et al., 2020; Toubiana et al., 2020, 2021).

Inflammatory features of MIS-C are in part overlapping with those of both KD and acute SARS-CoV-2 infection in children, as well as severe COVID-19 in adults (Carter et al., 2020; Consiglio et al., 2020; Datta et al., 2020; Gruber et al., 2020). Very high levels of C-reactive protein (CRP), Procalcitonin (PCT) and IL-6, might reflect a strong immunological response to a pathogenic SARS-CoV-2 superantigen (Cheng et al., 2020). Autoimmune features can also be found in MIS-C patients (Gruber et al., 2020).

Moreover, since July 2021, myocarditis and pericarditis are considered as an adverse reaction that may occur rarely following vaccination. A method of predicting the risk of myocarditis and pericarditis after vaccination against SARS-CoV-2 as thus needed.

Finally, the SARS-CoV-2 infection can cause severe or critical form of COVID-19. This prediction could be a useful tool to categorize disease severity and help in therapeutic decision involving immunomodulatory drugs.

### SUMMARY OF THE INVENTION

The present invention is defined by the claims. In particular, the present invention relates to methods of predicting multisystem inflammatory syndrome (MIS-C) with severe myocarditis in subjects suffering from a SARS-CoV-2 infection or disease severity following SARS-CoV-2 infection or myocarditis post-vaccination against SARS-CoV-2.

### DETAILED DESCRIPTION OF THE INVENTION

SARS-CoV-2 infection in children is generally milder than in adults, yet a proportion of cases result in hyperinflammatory conditions often including myocarditis with cardiac dysfunction. To better understand these cases, the inventors applied a multi-parametric approach to the study of blood cells of 56 children hospitalized with suspicion of SARS-CoV-2 infection. The most severe forms of MIS-C (multisystem inflammatory syndrome in children related to SARS-CoV-2), that resulted in myocarditis, were characterized by elevated levels of pro-angiogenesis cytokines and several chemokines. This phenotype was associated with TNF-α signaling, sustained NF-κB signaling in monocytic/dendritic cells, alongside increased HIF-la and VEGF signaling. Single-cell transcriptomic analyses identified a unique monocyte/dendritic cell gene signature that correlated with the occurrence of myocarditis, characterized by decreased gene expression of NF-κB inhibitors, a weak response to type-I and type-II interferons, increased TNF-α signaling, hyperinflammation and response to oxidative stress, providing potential for a better understanding of disease pathophysiology.

The inventors have also studied the severity of SARS-CoV-2 infection and they have determined a method of predicting the severe form of COVID-19.

Finally, the inventors have studied the side effect of vaccination against SARS-CoV-2 and they have determined a method of predicting of myocarditis following vaccination against SARS-CoV-2.

### Prediction of Multisystem Inflammatory Syndrome (MIS-C) with Severe Myocarditis

The present invention relates to a method of predicting whether a subject suffering from a SARS-CoV-2 infection is at risk of having a multisystem inflammatory syndrome (MIS-C) with severe myocarditis comprising determining the expression level in a sample obtained from the subject of at least one gene selected from the group consisting of:
RETN
CLU
CAPNS1
S100A8
PPBP
CTSA
PF4
PGD
P2RX1
S100A12
IFNGR2
TOP1MT
SLC25A37
VAT1
RBM3
CTSD
PGPEP1
TPST1
RPS9
GADD45GIP1
GAPDH
ALOX5AP
SH3BGRL3
PFDN1
LGALS1
PHC2
ATF4
RAC1
RPL6
LAPTM5
RPL38
SLC44A1
GMFB
HADHB
NAMPT
STK24
VIM
FTL
NDUFB9
RNF24
MMP24OS
GLUL
DAZAP2
RNF141
AREG
SPI1
CCDC69
APLP2
S100A6
SMIM3
RPL22
RPL7
CD63
LEPROT
RPS4Y1
CTSB
ASPH
ARL8A
ANPEP
BNIP2
ATP6V1F
BACH1
RPL37
ATP6V0B
POLR2L
ZYX
GSTO1
S100A10
GNG5
RPL35A
CHMP4B
QSOX1
FOXO3
CSGALNACT2
RUNX1
ASAH1
RFLNB
TNNT1
BRI3
RPL37A
SDCBP
AATK
CCDC71L
CARD19
TPD52L2
ADAM9
RPL21
TMEM167B
MBOAT7
ADD3
TIMP2
SRA1
THBD
CD9
ZFAND3
QKI
IL1R1
CXXC5
NRIP1
FBP1
CMTM6
SIRPA
C5AR1
TMA7
IRF2BP2
PHLDA1
TLNRD1
SLC6A6
FNDC3B
ADAP2
FAM49B
FAM20C
KRT10
HBEGF
RIT1
FCAR
   wherein the determined level indicates the risk of having a multisystem inflammatory syndrome (MIS-C) with severe myocarditis.

As used herein, the term “Severe Acute Respiratory Syndrome coronavirus 2” or “SARS-CoV-2” has its general meaning in the art and refers to the strain of coronavirus that causes coronavirus disease 2019 (“COVID-19”), a respiratory syndrome that manifests a clinical pathology resembling mild upper respiratory tract disease (common cold-like symptoms) and occasionally severe lower respiratory tract illness and extra-pulmonary manifestations leading to multi-organ failure and death.

As used herein, the term “multisystem inflammatory syndrome” or “MIS-C” has its general meaning in the art and refers to the inflammatory syndrome described in Whittaker, E., Bamford, A., Kenny, J., Kaforou, M, Jones, C. E., Shah, P., Ramnarayan, P., Fraisse, A., Miller, O., Davies, P., et al. (2020). Clinical Characteristics of 58 Children With a Pediatric Inflammatory Multisystem Syndrome Temporally Associated With SARS-CoV-2. JAMA 324, 259-269. The term is also known as “Pediatric Inflammatory Multisystem Syndrome Temporally Associated with SARS-CoV-2” or “PIMS-TS”. MIS-C is a condition where different body parts can become inflamed, including the heart, lungs, kidneys, brain, skin, eyes, or gastrointestinal organs. Subjects with MIS-C may have a fever and various symptoms, including abdominal (gut) pain, vomiting, diarrhea, neck pain, rash, bloodshot eyes, or feeling extra tired.

As used herein, the term “myocarditis” has its general meaning in the art and refers to the inflammation of the heart muscle (myocardium). Myocarditis generally reduces cardiac ability to pump and can cause rapid or abnormal heart rhythms (arrhythmias). As used, herein, the term “severe myocarditis” refers to myocarditis with acute heart failure that requires intensive care treatment (e.g. treatment in an intensive care unit).

Prediction of Myocarditis Post-Vaccination Against SARS-CoV-2

The present invention also relates to a method of predicting whether a subject is at risk of having myocarditis post-vaccination against SARS-CoV-2 comprising determining the expression level in a sample obtained from the subject of at least one gene selected from the group consisting of:
RETN
CLU
CAPNS1
S100A8
PPBP
CTSA
PF4
PGD
P2RX1
S100A12
IFNGR2
TOP1MT
SLC25A37
VAT1
RBM3
CTSD
PGPEP1
TPST1
RPS9
GADD45GIP1
GAPDH
ALOX5AP
SH3BGRL3
PFDN1
LGALS1
PHC2
ATF4
RAC1
RPL6
LAPTM5
RPL38
SLC44A1
GMFB
HADHB
NAMPT
STK24
VIM
FTL
NDUFB9
RNF24
MMP24OS
GLUL
DAZAP2
RNF141
AREG
SPI1
CCDC69
APLP2
S100A6
SMIM3
RPL22
RPL7
CD63
LEPROT
RPS4Y1
CTSB
ASPH
ARL8A
ANPEP
BNIP2
ATP6V1F
BACH1
RPL37
ATP6V0B
POLR2L
ZYX
GSTO1
S100A10
GNG5
RPL35A
CHMP4B
QSOX1
FOXO3
CSGALNACT2
RUNX1
ASAH1
RFLNB
TNNT1
BRI3
RPL37A
SDCBP
AATK
CCDC71L
CARD19
TPD52L2
ADAM9
RPL21
TMEM167B
MBOAT7
ADD3
TIMP2
SRA1
THBD
CD9
ZFAND3
QKI
IL1R1
CXXC5
NRIP1
FBP1
CMTM6
SIRPA
C5AR1
TMA7
IRF2BP2
PHLDA1
TLNRD1
SLC6A6
FNDC3B
ADAP2
FAM49B
FAM20C
KRT10
HBEGF
RIT1
FCAR
   wherein the determined level indicates the risk of having a myocarditis post-vaccination against SARS-CoV-2.

As used herein, the term “myocarditis post-vaccination against SARS-CoV-2” refers to the inflammation of the heart muscle (myocardium) after a vaccination against SARS-CoV-2. Myocarditis reduces cardiac ability to pump and can cause rapid or abnormal heart rhythms (arrhythmias). As used, herein, the term “severe myocarditis post-vaccination against SARS-CoV-2” refers to myocarditis, post-vaccination against SARS-CoV-2, that requires intensive care treatment (e.g. treatment in an intensive care unit).

As used herein, the terms “vaccine” or “vaccination” have their general meaning in the art and refer to a therapy consisting in stimulating the immune system so as to obtain a specific response from the body against an antigen, whether viral, bacterial, cellular or even molecular. Vaccines are obtained from harmless strains of viruses or bacteria, purified antigens or antigenic analogues. They are commonly used in prevention to prevent an individual from developing a disease, but they can also be used once the pathology has been declared, in order to direct the immune response against an invader.

Prediction of Severe or Critical Form of COVID-19

The present invention also relates to a method of predicting whether a subject suffering from a SARS-CoV-2 infection is at risk of having a severe or critical form of COVID-19 comprising determining the expression level in a sample obtained from the subject of at least one gene selected from the group consisting of:
RETN
CLU
CAPNS1
S100A8
PPBP
CTSA
PF4
PGD
P2RX1
S100A12
IFNGR2
TOP1MT
SLC25A37
VAT1
RBM3
CTSD
PGPEP1
TPST1
RPS9
GADD45GIP1
GAPDH
ALOX5AP
SH3BGRL3
PFDN1
LGALS1
PHC2
ATF4
RAC1
RPL6
LAPTM5
RPL38
SLC44A1
GMFB
HADHB
NAMPT
STK24
VIM
FTL
NDUFB9
RNF24
MMP24OS
GLUL
DAZAP2
RNF141
AREG
SPI1
CCDC69
APLP2
S100A6
SMIM3
RPL22
RPL7
CD63
LEPROT
RPS4Y1
CTSB
ASPH
ARL8A
ANPEP
BNIP2
ATP6V1F
BACH1
RPL37
ATP6V0B
POLR2L
ZYX
GSTO1
S100A10
GNG5
RPL35A
CHMP4B
QSOX1
FOXO3
CSGALNACT2
RUNX1
ASAH1
RFLNB
TNNT1
BRI3
RPL37A
SDCBP
AATK
CCDC71L
CARD19
TPD52L2
ADAM9
RPL21
TMEM167B
MBOAT7
ADD3
TIMP2
SRA1
THBD
CD9
ZFAND3
QKI
IL1R1
CXXC5
NRIP1
FBP1
CMTM6
SIRPA
C5AR1
TMA7
IRF2BP2
PHLDA1
TLNRD1
SLC6A6
FNDC3B
ADAP2
FAM49B
FAM20C
KRT10
HBEGF
RIT1
FCAR
   wherein the determined level indicates the risk of having a severe or critical form of COVID-19.

As used herein, the term “severe or critical form of COVID-19” refers to the progression of the disease to acute respiratory distress syndrome (ARDS), accountable for high mortality related to the damages of the alveolar lumen. Numerous patients with ARDS secondary to COVID-19 develop life-threatening thrombotic complications. More precisely severe form of COVID-19 can lead to critical illness, with acute respiratory distress (ARDS) and multiorgan failure as its primary complications, eventually followed by intravascular coagulopathy. Critical form of COVID-19 also relates to a patient meeting any of the following criteria: respiratory failure (defined as any of)—severe respiratory failure (PaO2/FiO2), deteriorating despite non-invasive forms of respiratory support (i.e. noninvasive ventilation (NIV), or high-flow nasal oxygen (HFNO)), requiring mechanical ventilation, hypotension or shock, impairment of consciousness or other organ failure respiratory distress syndrome).

Method of the Present Invention

As used herein, the terms “subject” or “patient” denote a mammal, such as a rodent, a feline, a canine, and a primate. Particularly, the subject according to the invention is a human. In some embodiments, the subject is a human infant. In some embodiments, the subject is a human child. In some embodiments, the subject is a premature human infant or human new-born. In some embodiments, the subject is a human teenager. In some embodiments, the subject is a human adult. In some embodiments, the subject is an elderly human. In some embodiments, the patient is less than 15 years old. In some embodiments, the patient is less than 10 years old. In some embodiments, the patient is less than 7 years old. In some embodiments, the patient is less than 5 years old. In some embodiments, the patient is less than 3 years old. In some embodiments, the patient is an adult. In some embodiments, the subject is more than 15 years old. In some embodiments, the subject is more than 20 years old. In some embodiments, the subject is more than 25 years old. In some embodiments, the subject is more than 30 years old. In some embodiments, the subject is more than 35 years old.

In some embodiment, the subject suffers from a SARS-CoV-2 infection. In some embodiment, the subject will have a risk of having a severe or critical form of COVID-19.

As used herein, the term “risk” in the context of the present invention, relates to the probability that an event will occur over a specific time period and can mean a subject's “absolute risk” or “relative risk”. Absolute risk can be measured with reference to either actual observation post-measurement for the relevant time cohort, or with reference to index values developed from statistically valid historical cohorts that have been followed for the relevant time period. Relative risk refers to the ratio of absolute risks of a subject compared either to the absolute risks of low risk cohorts or an average population risk, which can vary by how clinical risk factors are assessed. Odds ratios, the proportion of positive events to negative events for a given test result, are also commonly used (odds are according to the formula p/(l−p) where p is the probability of event and (1−p) is the probability of no event) to no-conversion. “Risk evaluation,” or “evaluation of risk” in the context of the present invention encompasses making a prediction of the probability, odds, or likelihood that an event or disease state may occur, the rate of occurrence of the event or conversion from one disease state to another. Risk evaluation can also comprise prediction of future clinical parameters, traditional laboratory risk factor values, or other indices of relapse, either in absolute or relative terms in reference to a previously measured population. The methods of the present invention may be used to make continuous or categorical measurements of the risk of conversion, thus diagnosing and defining the risk spectrum of a category of subjects defined as being at risk of conversion. In the categorical scenario, the invention can be used to discriminate between normal and other subject cohorts at higher risk.

In the present specification, the name of each of the various genes of interest refers to the internationally recognised name of the corresponding gene, as found in internationally recognised gene sequences and protein sequences databases, including in the database from the HUGO Gene Nomenclature Committee that is available notably at the following Internet address: http://www.gene.ucl.ac.uk/nomenclature/index.html. In the present specification, the name of each of the various genes of interest may also refer to the internationally recognised name of the corresponding gene, as found in the internationally recognised gene sequences and protein sequences database Genbank. Through these internationally recognised sequence databases, the nucleic acid and the amino acid sequences corresponding to each of the biological marker of interest described herein may be retrieved by the one skilled in the art.

In some embodiments, the sample is a blood sample. As used herein, the term “blood sample” refers to a whole blood sample, serum sample and plasma sample. A blood sample may be obtained by methods known in the art including venipuncture or a finger stick. Serum and plasma samples may be obtained by centrifugation methods known in the art. The sample may be diluted with a suitable buffer before conducting the assay.

In some embodiments, the sample is a PBMC sample. The term “PBMC” or “peripheral blood mononuclear cells” or “unfractionated PBMC”, as used herein, refers to whole PBMC, i.e. to a population of white blood cells having a round nucleus, which has not been enriched for a given sub-population. Typically, the PBMC sample according to the invention has not been subjected to a selection step to contain only adherent PBMC (which consist essentially of >90% monocytes) or non-adherent PBMC. A PBMC sample according to the invention therefore contains lymphocytes (B cells, T cells, ILCs cells, and NKT cells), monocytes, and precursors thereof. Typically, these cells can be extracted from whole blood using Ficoll, a hydrophilic polysaccharide that separates layers of blood, with the PBMC forming a cell ring under a layer of plasma. Additionally, PBMC can be extracted from whole blood using a hypotonic lysis buffer which will preferentially lyse red blood cells. Such procedures are known to the expert in the art.

In some embodiments, the sample is any sample containing immune cells. As used herein, the term “immune cell” has its general meaning in the art and refers to the cells of the immune system that can be categorized as lymphocytes (T-cells, B-cells and NK cells), neutrophils, and monocytes/macrophages. These are all types of white blood cells.

In some embodiments, the sample is immune cells from bronchioalveolar lavage (BAL). As used herein, the term “bronchioalveolar lavage” refers to a diagnostic method of the lower respiratory system in which a bronchoscope is passed through the mouth or nose into an appropriate airway in the lungs, with a measured amount of fluid introduced and then collected for examination.

In some embodiments, the sample is a sample of monocytes. As used herein, the term “monocyte” has its general meaning in the art and refers to a large mononuclear phagocyte of the peripheral blood. Monocytes vary considerably, ranging in size from 10 to 30 μm in diameter. The nucleus to cytoplasm ratio ranges from 2:1 to 1:1. The nucleus is often band shaped (horseshoe), or reniform (kidney-shaped). It may fold over on top of itself, thus showing brainlike convolutions. No nucleoli are visible. The chromatin pattern is fine, and arranged in skein-like strands. The cytoplasm is abundant and appears blue gray with many fine azurophilic granules, giving a ground glass appearance in Giemsa staining. Vacuoles may be present. More preferably, the expression of specific surface antigens is used to determine whether a cell is a monocyte cell. The main phenotypic markers of human monocyte cells include CD11b, CD11c, CD33 and CD115. Methods for isolating monocytes are well known in the art and typically include on cell sorting methods such as fluorescence activated cell sorting (FACS) or magnetic activated cell sorting (MACS). For instance non-monocytes cells may be magnetically labeled with a cocktail of monoclonal antibodies chosen antibodies directed against CD3, CD7, CD19, CD56, CD123 and CD235a. Kits for isolation of monocytes are commercially available from Miltenyi Biotec (Auburn, CA, USA), Stem Cells Technologies (Vancouver, Canada) or Dynal Bioech (Oslo, Norway).

The measurement of the expression level of the biomarker in the blood sample is typically carried-out using standard protocols known in the art.

Methods for determining the expression level of a gene product such as nucleic acid (e.g. RNA) are also well known in the art. Conventional methods typically involve polymerase chain reaction (PCR). For instance, U.S. Pat. Nos. 4,683,202, 4,683,195, 4,800,159, and 4,965,188 disclose conventional PCR techniques. PCR typically employs two oligonucleotide primers that bind to a selected target nucleic acid sequence. Primers useful in the present invention include oligonucleotides capable of acting as a point of initiation of nucleic acid synthesis within the target nucleic acid sequence. A primer can be purified from a restriction digest by conventional methods, or it can be produced synthetically. PCR involves use of a thermostable polymerase. The term “thermostable polymerase” refers to a polymerase enzyme that is heat stable, i.e., the enzyme catalyzes the formation of primer extension products complementary to a template and does not irreversibly denature when subjected to the elevated temperatures for the time necessary to effect denaturation of double-stranded template nucleic acids. Thermostable polymerases have been isolated from Thermus fiavus, T. ruber, T. thermophilus, T. aquaticus, T. lacteus, T. rubens, Bacillus stearothermophilus, and Methanothermus fervidus. Nonetheless, polymerases that are not thermostable also can be employed in PCR assays provided the enzyme is replenished. Typically, the polymerase is a Taq polymerase (i.e. Thermus aquaticus polymerase). Quantitative PCR is typically carried out in a thermal cycler with the capacity to illuminate each sample with a beam of light of a specified wavelength and detect the fluorescence emitted by the excited fluorophore. The thermal cycler is also able to rapidly heat and chill samples, thereby taking advantage of the physicochemical properties of the nucleic acids and thermal polymerase. In order to detect and measure the amount of amplicon (i.e. amplified target nucleic acid sequence) in the sample, a measurable signal has to be generated, which is proportional to the amount of amplified product. All current detection systems use fluorescent technologies. Some of them are non-specific techniques, and consequently only allow the detection of one target at a time. Alternatively, specific detection chemistries can distinguish between non-specific amplification and target amplification. These specific techniques can be used to multiplex the assay, i.e. detecting several different targets in the same assay. For example, SYBR® Green I probes, High Resolution Melting probes, TaqMan® probes, LNA® probes and Molecular Beacon probes can be suitable. TaqMan® probes are the most widely used type of probes. They were developed by Roche (Basel, Switzerland) and ABI (Foster City, USA) from an assay that originally used a radio-labelled probe (Holland et al. 1991), which consisted of a single-stranded probe sequence that was complementary to one of the strands of the amplicon. A fluorophore is attached to the 5′ end of the probe and a quencher to the 3′ end. The fluorophore is excited by the machine and passes its energy, via FRET (Fluorescence Resonance Energy Transfer) to the quencher. Traditionally, the FRET pair has been conjugated to FAM as the fluorophore and TAMRA as the quencher. In a well-designed probe, FAM does not fluoresce as it passes its energy onto TAMRA. As TAMRA fluorescence is detected at a different wavelength to FAM, the background level of FAM is low. The probe binds to the amplicon during each annealing step of the PCR. When the Taq polymerase extends from the primer which is bound to the amplicon, it displaces the 5′ end of the probe, which is then degraded by the 5′-3′ exonuclease activity of the Taq polymerase. Cleavage continues until the remaining probe melts off the amplicon. This process releases the fluorophore and quencher into solution, spatially separating them (compared to when they were held together by the probe). This leads to an irreversible increase in fluorescence from the FAM and a decrease in the TAMRA.

In some embodiments, the expression level of the gene is determined by RNA sequencing. As used, the term “RNA sequencing” refers to sequencing performed on RNA (or cDNA) instead of DNA, where typically, the primary goal is to measure expression levels, detect fusion transcripts, alternative splicing, and other genomic alterations that can be better assessed from RNA. RNA sequencing typically includes whole transcriptome sequencing or targeted exome sequencing. In some embodiments, targeted exome sequencing may be preferred. As used herein, the term “whole transcriptome sequencing” refers to the use of high throughput sequencing technologies to sequence the entire transcriptome in order to get information about a sample's RNA content. As used herein, the term “targeted exome sequencing” refers to the use of high throughput sequencing technologies to sequence some specific targeted sequencing. RNA sequencing can be done with a variety of platforms for example, the Genome Analyzer (Illumina, Inc., San Diego, Calif.) and the SOLiD™ Sequencing System (Life Technologies, Carlsbad, Calif), However, any platform useful for whole transcriptome sequencing may be used. Typically, the RNA is extracted, and ribosomal RNA may be deleted as described in U.S. Pub, No. 2011/0111409. cDNA sequencing libraries may be prepared that are directional and single or paired-end using commercially available kits such as the ScriptSeq™ M mRNA-Seq Library Preparation Kit (Epicenter Biotechnologies, Madison, Wis.). The libraries may also be barcoded for multiplex sequencing using commercially available barcode primers such as the RNA sequencing Barcode Primers from Epicenter Biotechnologies (Madison, Wis.). PCR is then carried out to generate the second strand of cDNA to incorporate the barcodes and to amplify the libraries. After the libraries are quantified, the sequencing libraries may be sequenced. Nucleic acid sequencing technologies are suitable methods for expression analysis. The principle underlying these methods is that the number of times a (DNA sequence is detected in a sample is directly related to the relative RNA levels corresponding to that sequence. These methods are sometimes referred to by the term Digital Gene Expression (DOE) to reflect the discrete numeric property of the resulting data. Early methods applying this principle were Serial Analysis of Gene Expression (SAGE) and Massively Parallel Signature Sequencing (MPSS). See, e.g., S. Brenner, et al., Nature Biotechnology 18(6):630-634 (2000). Typically RNA sequencing uses Next Generation Sequencing or NGS. As used herein, the term “Next Generation Sequencing” (NGS) refers to a relatively new sequencing technique as compared to the traditional Sanger sequencing technique. For review, see Shendure et al., Nature Biotech., 26(10): 1135-45 (2008), which is hereby incorporated by reference into this disclosure. For purpose of this disclosure, NGS may include cyclic array sequencing, microelectrophoretic sequencing, sequencing by hybridization, among others. By way of example, in a typical NGS using cyclic-array methods, genomic DNA or cDNA library is first prepared, and common adaptors may then be ligated to the fragmented genomic DNA or cDNA. Different protocols may be used to generate jumping libraries of mate-paired tags with controllable distance distribution. An array of millions of spatially immobilized PCR colonies or “polonies” is generated with each polonies consisting of many copies of a single shotgun library fragment. Because the polonies are tethered to a planar array, a single microliter-scale reagent volume can be applied to manipulate the array features in parallel, for example, for primer hybridization or for enzymatic extension reactions. Imaging-based detection of fluorescent labels incorporated with each extension may be used to acquire sequencing data on all features in parallel. Successive iterations of enzymatic interrogation and imaging may also be used to build up a contiguous sequencing read for each array feature.

Typically, the higher is the expression level of the gene, the higher is the risk of having MIS-C with severe myocarditis. Thus, in some embodiments, high level of the gene indicate a high risk of having MIS-C with severe myocarditis.

Typically, the higher is the expression level of the gene, the higher is the risk of having a severe or critical form of COVID-19. Thus, in some embodiments, high level of the gene indicate a high risk of having a severe or critical form of COVID-19.

Typically, the higher is the expression level of the gene, the higher is the risk of having myocarditis post-vaccination against SARS-CoV-2. Thus, in some embodiments, high level of the gene indicate a high risk of having myocarditis post-vaccination against SARS-CoV-2.

As used herein, the term “high” refers to a measure that is greater than normal, greater than a standard such as a predetermined reference value or a subgroup measure or that is relatively greater than another subgroup measure. For example, a high expression level refers to a expression level that is greater than a normal expression level. A normal expression level may be determined according to any method available to one skilled in the art. High expression level may also refer to a level that is equal to or greater than a predetermined reference value, such as a predetermined cutoff. High expression level may also refer to an expression level wherein a high level subgroup has relatively greater expression levels of than another subgroup. For example, without limitation, according to the present specification, two distinct patient subgroups can be created by dividing samples around a mathematically determined point, such as, without limitation, a median, thus creating a subgroup whose measure is high (i.e., higher than the median) and another subgroup whose measure is low. In some cases, a “high” level may comprise a range of level that is very high and a range of level that is “moderately high” where moderately high is a level that is greater than normal, but less than “very high”.

As used herein, the term “low” refers to a level that is less than normal, less than a standard such as a predetermined reference value or a subgroup measure that is relatively less than another subgroup level. For example, low expression level means an expression level that is less than a normal level of in a particular set of samples of patients. A normal expression level measure may be determined according to any method available to one skilled in the art. Low expression level may also mean a level that is less than a predetermined reference value, such as a predetermined cutoff. Low expression level may also mean a level wherein a low level subgroup is relatively lower than another subgroup. For example, without limitation, according to the present specification, two distinct patient subgroups can be created by dividing samples around a mathematically determined point, such as, without limitation, a median, thus creating a group whose measure is low (i.e., less than the median) with respect to another group whose measure is high (i.e., greater than the median).

In some embodiments, the method of the present invention further comprises comparing the expression level with a predetermined reference value wherein detecting a difference between the expression level and the predetermined reference value indicates the risk of a MIS-C with severe myocarditis or a severe or critical form of COVID-19 or myocarditis post-vaccination against SARS-CoV-2. Typically, when the expression level is higher than the predetermined reference value, then it is concluded that the subject has a high risk of having a MIS-C with severe myocarditis whereas when the expression level is lower than the predetermined reference value, then it is concluded that the subject has a low risk of having a MIS-C with severe myocarditis. Typically, when the expression level is higher than the predetermined reference value, then it is concluded that the subject has a high risk of having a severe or critical form of COVID-19 or myocarditis post-vaccination against SARS-CoV-2 whereas when the expression level is lower than the predetermined reference value, then it is concluded that the subject has a low risk of having a severe or critical form of COVID-19 or myocarditis post-vaccination against SARS-CoV-2

In some embodiments, the predetermined reference value is a threshold value or a cut-off value. Typically, a “threshold value” or “cut-off value” can be determined experimentally, empirically, or theoretically. A threshold value can also be arbitrarily selected based upon the existing experimental and/or clinical conditions, as would be recognized by a person of ordinary skilled in the art. For example, retrospective measurement in properly banked historical subject samples may be used in establishing the predetermined reference value. The threshold value has to be determined in order to obtain the optimal sensitivity and specificity according to the function of the test and the benefit/risk balance (clinical consequences of false positive and false negative). Typically, the optimal sensitivity and specificity (and so the threshold value) can be determined using a Receiver Operating Characteristic (ROC) curve based on experimental data. For example, after determining the quantification of the selected population in a group of reference, one can use algorithmic analysis for the statistic treatment of the expression levels determined in samples to be tested, and thus obtain a classification standard having significance for sample classification. The full name of ROC curve is receiver operator characteristic curve, which is also known as receiver operation characteristic curve. It is mainly used for clinical biochemical diagnostic tests. ROC curve is a comprehensive indicator that reflects the continuous variables of true positive rate (sensitivity) and false positive rate (1−specificity). It reveals the relationship between sensitivity and specificity with the image composition method. A series of different cut-off values (thresholds or critical values, boundary values between normal and abnormal results of diagnostic test) are set as continuous variables to calculate a series of sensitivity and specificity values. Then sensitivity is used as the vertical coordinate and specificity is used as the horizontal coordinate to draw a curve. The higher the area under the curve (AUC), the higher the accuracy of diagnosis. On the ROC curve, the point closest to the far upper left of the coordinate diagram is a critical point having both high sensitivity and high specificity values. The AUC value of the ROC curve is between 1.0 and 0.5. When AUC>0.5, the diagnostic result gets better and better as AUC approaches 1. When AUC is between 0.5 and 0.7, the accuracy is low. When AUC is between 0.7 and 0.9, the accuracy is moderate. When AUC is higher than 0.9, the accuracy is high. This algorithmic method is preferably done with a computer. Existing software or systems in the art may be used for the drawing of the ROC curve, such as: MedCalc 9.2.0.1 medical statistical software, SPSS 9.0, ROCPOWER. SAS, DESIGNROC.FOR, MULTIREADER POWER. SAS, CREATE-ROC.SAS, GB STAT VI0.0 (Dynamic Microsystems, Inc. Silver Spring, Md., USA), etc.

In some embodiments, one or more expression level(s) is determined. In some embodiments, the expression level of 2; 3; 4; 5; 6; 7; 8; 9; 10; 11; 12; 13; 14; 15; 16; 17; 18; 19; 20; 21; 22; 23; 24; 25; 26; 27; 28; 29; 30; 31; 32; 33; 34; 35; 36; 37; 38; 39; 40; 41; 42; 43; 44; 45; 46; 47; 48; 49; 50; 51; 52; 53; 54; 55; 56; 57; 58; 59; 60; 61; 62; 63; 64; 65; 66; 67; 68; 69; 70; 71; 72; 73; 74; 75; 76; 77; 78; 79; 80; 81; 82; 83; 84; 85; 86; 87; 88; 89; 90; 91; 92; 93; 94; 95; 96; 97; 98; 99; 100; 101; 102; 103; 104; 105; 106; 107; 108; 109; 110; 111; 112; 113; 114; 115; or 116 genes are determined.

In some embodiments, the expression levels of 25 genes are determined. In some embodiments, the expression levels of:
RETN
CLU
CAPNS1
S100A8
PPBP
CTSA
PF4
PGD
P2RX1
S100A12
IFNGR2
TOP1MT
SLC25A37
VAT1
RBM3
CTSD
PGPEP1
TPST1
RPS9
GADD45GIP1
GAPDH
ALOX5AP
SH3BGRL3
PFDN1
LGALS1
   are determined in the sample obtained from the subject a child suffering from a SARS-CoV-2 infection.

In some embodiments, a score is calculated. The advantage of said score is to make easier the comparison step with the predetermined reference levels that may be expressed as “cut-off values” as described above. As used herein, the term “score” as used herein refers to a numerical value which is linked or based on a specific feature, e.g. the expression level of the gene. For instance example of a score is e.g. a Z score that quantifies how much the expression levels of a particular set of genes differs from the expression levels that were obtained from the same set of genes in reference samples. It is known to a person skilled in the art how such a Z score can be calculated. Typically, the Z-score may be determined as described in the EXAMPLE.

In some embodiments, the score is determined by an algorithm. Thus in some embodiments, the method of the invention thus comprises the use of an algorithm. As used herein, the term “algorithm” encompasses any formula, model, mathematical equation, algorithmic, analytical or programmed process, or statistical technique or classification analysis that takes one or more inputs or parameters, whether continuous or categorical, and calculates an output value (e.g. a score). Examples of algorithms include but are not limited to ratios, sums, regression operators such as exponents or coefficients, biomarker value transformations and normalizations (including, without limitation, normalization schemes that are based on clinical parameters such as age, gender, ethnicity, etc.), rules and guidelines, statistical classification models, and neural networks trained on populations. In some embodiments, the algorithm is a classification algorithm typically selected from Multivariate Regression Analysis, Linear Discriminant Analysis (LDA), Topological Data Analysis (TDA), Neural Networks, Support Vector Machine (SVM) algorithm and Random Forests algorithm (RF).

In some embodiments, the method of the present invention thus comprises a) determining the expression level of one or more gene(s) in the sample obtained from the subject; b) implementing an algorithm on data comprising the expression level so as to obtain an algorithm output; c) determining the risk of having a MIS-C with severe myocarditis or a severe or critical form of COVID-19 or myocarditis post-vaccination against SARS-CoV-2 from the output obtained at step c).

The method is thus particularly suitable for determining whether the subject is eligible to a particular therapy. In some embodiments, the subject being at risk of having a MIS-C with a severe myocarditis or a severe or critical form of COVID-19 or myocarditis post-vaccination against SARS-CoV-2 may be administered with a corticosteroid, IVIG and/or with a TNF blocking agent. More particularly, the subject considered being at risk of having a MIS-C with a severe myocarditis or a severe or critical form of COVID-19 or myocarditis post-vaccination against SARS-CoV-2 may be administered with a corticosteroid, in combination with IVIG. In some embodiments, the subject having a low risk of having a MIS-C with a severe myocarditis or a severe or critical form of COVID-19 or myocarditis post-vaccination against SARS-CoV-2 may be administered with a corticoid alone.

Thus in some embodiments, the subject is administered with a therapeutically effective amount of IVIG. As used herein the term “intravenous immunoglobulin” or “IVIG” refers to a blood product that contains the pooled immunoglobulin G (IgG) immunoglobulins from the plasma of a large number (often more than a thousand) of blood donors. Typically containing more than 95% unmodified IgG, which has intact Fc-dependent effector functions, and only trace amounts of immunoglobulin A (IgA) or immunoglobulin M (IgM), IVIGs are sterile, purified IgG products used in treating certain medical conditions. Although the word “intravenous” typically indicates administration by intravenous injection, the term “IVIG” or “IVIG composition” as used in this patent application also encompasses an IgG composition that is formulated for administration by additional routes, including subcutaneous or intranasal administration.

Thus in some embodiments, the subject is administered with a therapeutically effective amount of a corticosteroid. As used, the term “corticosteroid” has its general meaning in the art and refers to class of active ingredients having a hydrogenated cyclopentoperhydrophenanthrene ring system endowed with an anti-inflammatory activity. Corticosteroid drugs typically include cortisone, cortisol, hydrocortisone (11β,17-dihydroxy, 21-(phosphonooxy)-pregn-4-ene, 3,20-dione disodium), dihydroxycortisone, dexamethasone (21-(acetyloxy)-9-fluoro-1β,17-dihydroxy-16α-m-ethylpregna-1,4-diene-3,20-dione), and highly derivatized steroid drugs such as beconase (beclomethasone dipropionate, which is 9-chloro-11-β, 17,21, trihydroxy-16β-methylpregna-1,4 diene-3,20-dione 17,21-dipropionate). Other examples of corticosteroids include flunisolide, prednisone, prednisolone, methylprednisolone, triamcinolone, deflazacort and betamethasone. corticosteroids, for example, cortisone, hydrocortisone, methylprednisolone, prednisone, prednisolone, betamethesone, beclomethasone dipropionate, budesonide, dexamethasone sodium phosphate, flunisolide, fluticasone propionate, triamcinolone acetonide, betamethasone, fluocinolone, fluocinonide, betamethasone dipropionate, betamethasone valerate, desonide, desoximetasone, fluocinolone, triamcinolone, triamcinolone acetonide, clobetasol propionate, and dexamethasone.

Thus in some embodiments, the subject is administered with a therapeutically effective amount of a TNF blocking agent. As used herein, the term “TNFα blocking agent” or “TBA”, it is herein meant a biological agent which is capable of neutralizing the effects of TNFα. Said agent is a preferentially a protein such as a soluble TNFα receptor, e.g. Pegsunercept, or an antibody. In some embodiments, the TBA is a monoclonal antibody having specificity for TNFα or for TNFα receptor. In some embodiments, the TBA is selected in the group consisting of Etanercept (Enbrel®), Infliximab (Remicade®), Adalimumab (Humira®), Certolizumab pegol (Cimzia®), and golimumab (Simponi®). Recombinant TNF-receptor based proteins have also been developed (e.g. etanercept, a recombinant fusion protein consisting of two extracellular parts of soluble TNFα receptor 2 (p75) joined by the Fc fragment of a human IgG1 molecule). A pegylated soluble TNF type 1 receptor can also be used as a TNF blocking agent. Additionally, thalidomide has been demonstrated to be a potent inhibitor of TNF production. TNFα blocking agents thus further include phosphodiesterase 4 (IV) inhibitor thalidomide analogues and other phosphodiesterase IV inhibitors. As used herein, the term “etanercept” or “ETA” denotes the tumor necrosis factor-alpha (TNFα) antagonist used for the treatment of rheumatoid arthritis. The term “etanercept” (ETA, ETN, Enbrel) is a recombinant TNF-receptor IgG-Fc-fusion protein composed of the p75 TNF receptor genetically fused to the Fc domain of IgG1. Etanercept neutralizes the proinflammatory cytokine tumor necrosis factor-α (TNFα) and lymphotoxin-α (Batycka-Baran et al., 2012).

The invention will be further illustrated by the following figures and examples. However, these examples and figures should not be interpreted in any way as limiting the scope of the present invention.

### FIGURES

FIG. 1: Identification of genes specifically regulated in children of the MIS-C_MYO (CoV2+) group from SC-RNA-SEQ and validated by bulk RNA-SEQ. A. Box plots of the expression of the 116 genes validated in C, calculated as a signature score. SignatureScore represents for each sample the mean z-score of the 116 genes selected in B in the bulk-RNASEQ dataset. B. Boxplots showing the signature score computed on the expression of the top 25 genes, as ranked in FIG. 2A, in the bulk-RNA-SEQ dataset. A&B Each dot represents a sample. Boxes range from the 25th to the 75th percentiles. The upper and lower whiskers extend from the box to the largest and smallest values respectively. Any samples with a value at most ×1.5 the inter-quartile range of the hinge is considered an outlier and plotted individually.

FIG. 2: Signature Score and top genes correlated with the occurrence of myocarditis in MIS-C (CoV2+). A. Top: Barplot of the RankingScore of each gene. Genes are ranked based on their RankingScore as explained in Methods B. Curated analyses based on literature regarding the known links between the top 25 genes, and inflammatory response, myocarditis, oxidative stress, TNF-alpha, NF-kB, MIS-C or Kawasaki disease, SARS-CoV-2 and/or COVID-19. On the Y axis, genes are positioned from top to bottom following their rank (Top=ranked 1). A dot means strong links between the gene and the pathway (on the X axis) were found in peer-reviewed scientific publications.

FIG. 3: Test of the 116 gene signature on myocarditis post SARS-CoV-2 and myocarditis post SARS-CoV-2 vaccination in children and adults. A. Signature score of the 116 genes on data generated and published in the Med paper DOI: 10.1016/j.medj.2021.08.002. Bulk-RNA-seq on PBMCs. HC: Healthy Controls; MIS-C: Multisystem Inflammatory Syndrome in Children; MIS-C_MYO: Multisystem Inflammatory Syndrome in Children with severe myocarditis. B. Signature score of the 116 genes using Targeted RNA Next Generation Sequencing (Agilent technology (T-RNA-NGS)) on PBMCs extracted from children with MIS-C, MIS-C with severe myocarditis (MIS-C_MYO) and children who developed myocarditis following vaccination against SARS-CoV-2.

FIG. 4: Test of the 116 gene signature on severe and critical COVID-19 in children and adults. A. Signature score of the 116 genes using Targeted RNA Next Generation Sequencing (Agilent technology (T-RNA-NGS)) on whole blood cells extracted from adults with moderate COVID-19 (Moderate_COVID-19) and severe COVID-19 (Severe_COVID-19) (Data generated and analyzed by Menager's team). B. Signature score of the 116 genes on data generated and published in the Science paper DOI: 10.1126/science.abc6261. Bulk-RNA-Seq on PBMC from adult healthy controls (HC), adults with moderate COVID-19 (Moderate_COVID-19), adults with Severe COVID-19 (Severe_COVID-19) and adults with critical COVID-19 requiring Intensive Care Unit (ICU_COVID-19). C. Signature score of the 116 genes on data generated and published in the Nat Med paper DOI.org/10.1038/s41591-020-0901-9. Single-Cell-RNA-Seq on immune cells extracted from bronchioalveolar lavages from adults with moderate COVID-19 (Moderate_COVID-19), adults with Severe_COVID-19 (Severe_COVID-19) and adults with critical COVID-19 requiring Intensive Care Unit (ICU_COVID-19).

### EXAMPLE

### Methods

### Patients and Definitions

This prospective multicenter cohort study included children (age ≤18 years at the time of admission) suspected of infection with SARS-CoV-2 between Apr. 6, 2020 and May 30, 2020. Clinical aspects of 22 of the included patients were previously reported (Toubiana et al., 2020, 2021). Children admitted with fever in general pediatric wards or pediatric intensive care units of Tertiary French hospitals involved in the research program, suspected of SARS-CoV-2 related illness and who underwent routine nasopharyngeal swabs for SARS-CoV-2 RT-PCR (R-GENE, Argene, Biomerieux, Marcy l'Etoile) or SARS-CoV-2 IgG serology testing (Architect SARS-CoV-2 chemiluminescent microparticle immunoassay; Abbott Core Laboratory, IL, USA), were eligible. The study was approved by the Ethics Committee (Comité de Protection des Personnes Ouest IV, no DC-2017-2987). All parents provided written informed consent.

Case definition for pediatric COVID-19 acute infection was presence of fever, fatigue, neurological abnormalities, gastro-intestinal or respiratory signs, associated with a concomitant nasopharyngeal swab positive for SARS-CoV-2 RT-PCR, and absence of MIS-C criteria (Zimmermann and Curtis, 2020). Case definition for postacute hyperinflammatory illness was presence of fever, laboratory evidence of inflammation and clinically severe illness with multisystem involvement, during the SARS-CoV-2 epidemic period (Datta et al., 2020). This may include children with features of KD-like illness; criteria of the American Heart Association was used to define for complete (Fever >4 days and ≥4 principal criteria) or incomplete KD (Fever >4 days and 2 or 3 principal criteria, and without characteristics suggestive of another diagnosis) (McCrindle et al., 2017). Among cases with postacute hyperinflammatory illness, children with a positive SARS-CoV-2 testing (RT-PCR or serology) were considered to have MIS-C according to CDC and WHO criteria to define MIS-C (CDC, 2020). Patients with postacute hyperinflammatory illness, negative SARS-CoV-2 testing (RT-PCR or serology), and criteria for KD, were considered as patients with KD-like illness. Patients with MIS-C requiring intensive care, with elevated high-sensitivity cardiac troponin I levels (>26 ng/mL) and/or cardiac dysfunction (diastolic or systolic ventricular dysfunction at echocardiography), were considered to have MIS-C with severe myocarditis.

For each included patient, we collected demographic data, symptoms, results of SARS-CoV-2 testing and other laboratory tests, echocardiograms, and treatments. All patients with negative initial serology testing were retested after an interval of at least 3 weeks (Architect SARS-CoV-2 chemiluminescent microparticle immunoassay; Abbott Core Laboratory).

Healthy controls were recruited before the COVID-19 pandemic (before November 2019).

### Samples

For each patient and healthy donor, peripheral blood samples were collected on EDTA and lithium heparin tubes. After a centrifugation of the EDTA tube at 2300 rpm for 10 minutes, plasma was taken and stored at −80° C. before cytokine quantification. 300 μL of whole blood was taken from each lithium heparin tube and used for cell phenotyping by CyTOF. PBMCs were isolated from the remaining lithium heparin samples, frozen as described below and stored at −80° C. and were used for both bulk and single-cell RNAseq, as well as cell phenotyping by CyTOF.

### Isolation of PBMCs

Peripheral blood samples were collected on lithium heparin. PBMCs were isolated by density gradient centrifugation (2200 rpm without break for 30 minutes) using Ficoll (Eurobio Scientific, Les Ulis, France). After centrifugation, cells were washed with Phosphate-buffered saline (PBS) (Thermo Fisher scientific, Illkirch, France). The pellet was resuspended in PBS and cells were centrifuged at 1900 rpm for 5 minutes. Finally, the PBMC pellet was frozen in a medium containing 90% of Fetal Bovine Serum (FBS) (Gibco, Thermo Fisher scientific, Illkirch, France) and 10% of dimethyl sulfoxide (DMSO) (Sigma Aldrich, St. Quentin Fallavier, France).

### Cytokine Measurements

Prior to protein analysis plasma samples were treated in a BSL3 laboratory for viral decontamination using a protocol previously described for SARS-CoV (Darnell and Taylor, 2006), which we validated for SARS-CoV-2. Briefly, samples were treated with TRITON X100 (TX100) 1% (v/v) for 2 hrs at RT. IFNα2, IFNγ, IL-17A, (triplex) and IFNβ (single plex) protein plasma concentrations were quantified by Simoa assays developed with Quanterix Homebrew kits as previously described (Rodero et al., 2017). IL-6, TNFα, and IL-10 were measured with a commercial triplex assay (Quanterix). The limit of detection (LOD) of these assays were 0.6 pg/mL for IFNβ, 2 fg/mL for IFNα2, 0.05 pg/ml for IFNγ and 3 pg/mL for IL17A including the dilution factor. Additional plasma cytokines and chemokines (44 analytes) were measured with a commercial Luminex multi-analyte assay (Biotechne, R&D systems).

### Serology Assays

SARS-CoV-2 specific antibodies were quantified using assays previously described (Grzelak et al., 2020). Briefly, a standard ELISA assay using as target antigens the extracellular domain of the S protein in the form of a trimer (ELISA tri-S) and the S-Flow assay, which is based on the recognition of SARS-CoV-2 S protein expressed on the surface of 293T cells (293T-S), were used to quantify SARS-CoV-2 specific IgG and IgA subtypes in plasma. Assay characteristics including sensitivity and specificity were previously described (Grzelak et al., 2020).

### Cell Phenotyping

To perform high-dimensional immune profiling of PBMCs, we used the Maxpar® Direct™ Immune Profiling System (Fluidigm, Inc France) with a 30-marker antibody panel. Briefly, 3×10⁶ PBMCs resuspended in 300 μl of MaxPar Cell Staining Buffer were incubated for 20 minutes at room temperature after addition of 3 μL of 10 KU/mL heparin solution and 5 μl of Human TruStain FcX (Biolegend Europ, Netherland). Then 270 μL of the samples were directly added to the dry antibody cocktail during 30 minutes. 3 mL of MaxPar Water was added to each tube for an additional 10-min incubation. Three washes were performed on all the samples using MaxPar Cell Staining Buffer and they were fixed using 1.6% paraformaldehyde (Sigma-Aldrich, France). After one wash with MaxPar Cell Staining Buffer, cells were incubated one hour in Fix and Perm Buffer with 1:1000 of Iridium intercalator (pentamethylcyclopentadienyl-Ir (III)-dipyridophenazine, Fluidigm, Inc France). Cells were washed and resuspended at a concentration of 1 million cells per mL in Maxpar Cell Acquisition Solution, a high-ionic-strength solution, and mixed with 10% of EQ Beads immediately before acquisition.

Acquisition of the events was made on the Helios mass cytometer and CyTOF software version 6.7.1014 (Fluidigm, Inc Canada) at the “Plateforme de Cytométrie de la Pitié-Salpetriere (CyPS).” An average of 500,000 events were acquired per sample. Dual count calibration, noise reduction, cell length threshold between 10 and 150 pushes, and a lower convolution threshold equal to 10 were applied during acquisition. Mass cytometry standard files produced by the HELIOS were normalized using the CyTOF Software v. 6.7.1014. For data cleaning, 4 parameters (centre, offset, residual and width) are used to resolve ion fusion events (doublets) from single events from the Gaussian distribution generated by each event (Bagwell et al., 2020). Subsequent to data cleaning, the program produces new FCS files consisting of only intact live singlet cells. These data were analyzed in FlowJo v10.7.1 using 3 plugins (DownSampleV3, UMAP and FlowSOM) with R v4.0.2. To increase efficiency of the analysis, samples were downsampled to 50 000 cells, using the DownSample V3 plugin. All samples were concatenated and analyzed in an unsupervised manner. Anti-CD127 antibody had to be excluded due to poor staining. Clustering was performed using FlowSOM (Van Gassen et al., 2015). The number of clusters was set to forty-five in order to overestimate the populations and detect smaller subpopulations. Grid size of the self-organizing map was set to 20×20. Resulting clusters were annotated as cell populations following the kit manufacturer's instruction. When several clusters were identified as the same cell types, they were concatenated into a single cell population. For visualization purposes, UMAP was computed with the UMAP pluggin (McInnes et al.) with the following parameters: metric (Euclidean), nearest neighbors (15), minimum distance (0.5) and number of components (2). For the Whole blood samples, neutrophils were isolated, and the same process was repeated on the 3 markers of neutrophils (HLA-DR, CD66b and CD16) to obtain the neutrophils' UMAP.

### Single-Cell Transcriptomic (SC-RNA-SEQ)

SC-RNA-SEQ analyses were performed on frozen PBMCs isolated from heparin blood samples. PBMCs were thawed according to 10× Genomics protocol. The SC-RNA-SEQ libraries were generated using Chromium Single Cell 3′ Library & Gel Bead Kit v.3 (10× Genomics) according to the manufacturer's protocol. Briefly, cells were counted, diluted at 1000 cells/μL in PBS+0.04% and 20,000 cells were loaded in the 10× Chromium Controller to generate single-cell gel-beads in emulsion. After reverse transcription, gel-beads in emulsion were disrupted. Barcoded complementary DNA was isolated and amplified by PCR. Following fragmentation, end repair and A-tailing, sample indexes were added during index PCR. The purified libraries were sequenced on a Novaseq 6000 (Illumina) with 28 cycles of read 1, 8 cycles of i7 index and 91 cycles of read 2.

Sequencing reads were demultiplexed and aligned to the human reference genome (GRCh38, release 98, built from ensembl sources), using the CellRanger Pipeline v3.1. Unfiltered RNA UMI counts were loaded into Seurat v3.1 (Stuart et al., 2019) for quality control, data integration and downstream analyses. Apoptotic cells and empty sequencing capsules were excluded by filtering out cells with fewer than 500 features or a mitochondrial content higher than 20%. Data from each sample were log-normalized and scaled, before batch correction using Seurat's FindIntegratedAnchors. For computational efficiency, anchors for integration were determined using all control samples as reference and patient samples were projected onto the integrated controls space. On this integrated dataset, we computed the principal component analysis on the 2000 most variable genes. UMAP was carried out using the 20 most significant PCs, and community detection was performed using the graph-based modularity-optimization Louvain algorithm from Seurat's FindClusters function with a 0.8 resolution. Cell types labels were assigned to resulting clusters based on a manually curated list of marker genes as well as previously defined signatures of the well-known PBMC subtypes (Monaco et al., 2019). Despite filtering for high quality cells, five clusters out of the twenty-six stood out as poor quality clusters and were removed from further analysis, namely: one erythroid-cell contamination (cluster 7); one low UMI cluster from a single control (cluster 12); two clusters of proliferating cells originating from a patient with EBV co-infection (clusters 16 and 24) and one megakaryocytes cluster. In total 152,201 cells were kept for further analysis.

After extraction and reclustering of each cell population (Myeloid cells, T cells or B cells), differential expression was performed separately on all PBMCs, Myeloid cells, T cells or B cells. Differential expression testing was conducted using the FindMarkers function of Seurat on the RNA assay with default parameters. Genes with log(FC)>0.25 and adjusted p-values <=0.05 were selected as significant.

### Bulk RNA-Sequencing (Bulk-RNA-SEQ)

Bulk RNA-SEQ analyses were performed on frozen PBMCs extracted from heparin samples. RNA was extracted from PBMCs following the instructions of RNeasyR Mini kit (Qiagen, Courtaboeuf, France). To note, the optional step with the DNase was performed. RNA integrity and concentration were assessed by capillary electrophoresis using Fragment Analyzer (Agilent Technologies). RNAseq libraries were prepared starting from 100 ng of total RNA using the Universal Plus mRNA-Seq kit (Nugen) as recommended by the manufacturer. The oriented cDNA produced from the poly-A+ fraction was sequenced on a NovaSeq6000 from Illumina (Paired-End reads 100 bases+100 bases). A total of ˜50 millions of passing-filters paired-end reads was produced per library.

Paired-end RNA-seq reads were aligned to the human Ensembl genome [GRCh38.91] reference using Hisat2 (v2.0.4)(Kim et al., 2019) and counted using featureCounts from the Subread R package. The raw count matrix was analyzed using DESeq2 (version 1.28.1) (Love et al., 2014). No pre-filtering was applied to the data. Differential expression analysis was performed using the “DESeq” function with default parameters. For visualization and clustering, the data was normalized using the ‘variant stabilizing transformation’ method implemented in the “vst” function. Plots were generated using ggplot2 (version 3.3.2), and pheatmap (version 1.0.12).

During exploratory analyses, it was noted that the clustering was mainly driven by the sex of the patients. To remove this effect, it was included in the regression formula for DESeq (˜sex+groups), and then removed following vst transformation, using “removeBatchEffect” from the “limma” package (version 3.44.3).

### Gene Signature Analysis

To identify genes that could be used as markers of myocarditis in the SC-RNA-SEQ dataset, three initial strategies were used, all based on differential expression and selection of the upregulated genes. First, we performed the differential expression between MIS-C_MYO (CoV2⁺) samples and all other samples. Second, differential analysis was computed between MIS-C_MYO (CoV2⁺) and other samples with KD criteria (KD and MIS-C). In the last strategy, we selected genes that were upregulated between the MIS-C_MYO (CoV2⁺) and the CTL, but not upregulated in any other group compared to the CTL. These three strategies allowed us to identify 329 unique genes.

To further explore whether these genes could be considered as markers of myocarditis, we analyzed their expression profile in our bulk RNA-SEQ dataset. This dataset excluded samples from patients of the MIS-C_MYO (CoV2⁺) that were included in the SC-RNA-SEQ cohort. Vst-transformed counts were log 2-normalized and converted to z-score using the scale function in R (v 4.0.2). A GeneSCORE was computed for each group as the mean z-score of the samples of a group. Heatmaps representing this GeneSCORE_{group} were performed using pheatmap. Hierarchical clustering of the 329 previously identified genes was performed using the complete method on the distance measured using Pearson's correlation, as implemented by pheatmap. The hierarchical clustering was divided into 15 main clusters, 4 of which had the expected pattern of expression: Clusters that had a higher expression in MIS-C_MYO (CoV2⁺) than any other group were selected, resulting in 116 genes. A signature score for each sample was performed on these genes, corresponding to the mean expression (z-score) of these N genes in each sample (SignatureSCORE).

These genes were subsequently ranked based on the following equation:

RankingSCORE=GeneSCORE_{MIS-C_MYO (CoV2+)}−(GeneSCORE_{MIS-C (CoV2+)}) where the SCOREs represent the mean expression (z-score) in each disease groups, and the signature score was computed on the top 5, top 15 and top 25 genes.

### Quantification and Statistical Analysis

Cytokine heatmaps were made with Qlucore OMICS explore (version 3.5(26)) and dot plots with GraphPad Prism (version 8). Differential cytokines were included in the heat maps based on a 1.5 FC comparison between groups as indicated. Dot plot differences between each groups were identified by Kruskal-Wallis tests followed by post-hoc multiple comparison Dunn's test. Statistical tests for cellular composition analysis in both the CyTOF and SC datasets were performed in R v3.6.1. Kruskal-Wallis test followed by post-hoc multiple comparison Dunn's test was applied to assess differences in cell population proportions (*: p≤0.05; **: p≤0.01; ***: p≤0.001; ****: p≤0.0001).

Differential expression testing in the single cell dataset was conducted using the FindMarkers function in Seurat, with default Wilcoxon testing. P-values were controlled using Bonferroni correction. Genes with an absolute log(fold-change)≥0.25 and an adjusted p-value ≤0.05 were selected as differentially expressed. Volcano plots were plotted using the EnhancedVolcano package. Pathways analysis was performed using both the Ingenuity pathway analysis v57662101 software (IPA (QIAGEN Inc., https://www.qiagenbioinformatics.com/products/ingenuitypathway-analysis) and EnrichR (Chen et al., 2013; Kuleshov et al., 2016). Heatmaps were extracted from the comparison module in IPA. Pathways with a z-score lower than 2 or a Bonferroni-Hochberg corrected p-values were filtered out. Biocarta 2016, Reactome 2016 and Molecular Signature DataBase Hallmark 2020 (MSigDB Hallmark 2020) pathway enrichment analysis were performed using EnrichR. The TRRUST transcription factors 2019 (Han et al., 2018) used for the transcription factors enrichment analysis was performed using Enrich R.

### Results

### Clinical Description of the Cohort

The study cohort consisted of 56 children hospitalized during the first peak of the SARS-CoV-2 pandemic (from the 6^{th} of April to the 30^{th} of May), and 34 healthy controls (N=26 pediatric and N=8 adults) (data not shown). Among the 13 children with acute respiratory infection (data not shown), 9 had a confirmed SARS-CoV-2 infection (RT-PCR on nasopharyngeal aspiration or swab) (Acute-inf (CoV2⁺) group). Six out of these 9 cases had pneumonia, and one had an uncomplicated febrile seizure. One patient with a history of recent bone marrow transplantation for sickle cell disease, required intensive care support. The 4 other patients (Acute-inf (CoV2⁻) group) had pneumonia associated with a positive RT-PCR test for either Mycoplasma pneumoniae or rhinovirus/enterovirus, and negative RT-PCR for SARS-CoV-2. Fourty-three children displayed features of postacute hyperinflammatory illness (data not shown). Most (n=30) had a confirmed SARS-CoV-2 infection (positive for serum IgG, with 14 also positive for concomitant nasopharyngeal RT-PCR testing) and were therefore considered as cases of MIS-C (MIS-C (CoV2⁺) group); thirteen tested negative for SARS-CoV-2 and fulfilled clinical criteria for complete (n=6) or incomplete (n=7) Kawasaki disease (KD), and were therefore considered to have KD-like illness (KD (CoV2⁻) group) (data not shown). SARS-CoV-2 infection status of all samples was confirmed by specific antibody determination (both IgG and IgA) in the plasma, using ELISA and flow cytometry-based technics as described in the methods (data not shown). The 30 cases of MIS-C presented clinical features of KD, 14 of them fulfilled clinical criteria for a complete form of KD according to the American Heart Association (McCrindle et al., 2017). Of note, 21/30 cases had severe myocarditis (i.e. elevated high-sensitivity cardiac troponin I results, associated with ventricular dysfunction requiring intensive care support; MIS-C_MYO (CoV2⁺)). MIS-C cases had low lymphocyte counts and those with severe myocarditis had in addition abnormally increased neutrophil counts as compared to other groups, along with high levels of CRP, PCT, serum alanine transaminases (ALT) and ferritin (data not shown). All cases responded favorably to intravenous immunoglobulin injections (IVIG), associated in some (N=12, data not shown) with glucocorticosteroids. Multi-parametric analyses were performed at a median fever persistence of 9-10 days (data not shown).

### Elevated Inflammatory Cytokine Levels in Pediatric Acute Infection and Postacute Hyperinflammatory Conditions.

We investigated in all patients, plasma cytokine and chemokine levels by Luminex and Simoa assays. Hierarchical clustering analysis and stratification by patient groups revealed overall elevated levels of immune and inflammatory markers, with 40/46 measured proteins significantly elevated (q<0.05) as compared to healthy controls (data not shown). Twelve cytokines were found to be elevated in all groups of patients as compared to healthy controls (data not shown). High IL-8 and CXCL1 (data not shown) were more specific to children with acute infection. Cytokine levels did not significantly differ between children with acute infection with or without evidence of SARS-CoV-2 infection (data not shown). IFNγ, IFNα2, IL-17A, TNF-α, IL-10, Granzyme B, were higher in children with postacute hyperinflammation (MIS-C (CoV2⁺), MIS-C_MYO (CoV2⁺), and KD (CoV2⁻) groups), as compared to pediatric healthy donors (CTL) and patients with acute infections (Acute-inf (CoV2⁺) and Acute-inf (CoV2⁻) (data not shown). A slightly higher expression of IFNα2 and IL-17A was found in MIS-C without myocarditis (MIS-C (CoV-2⁺) patients). (data not shown). In contrast, 11 cytokines and chemokines (CSF2, CCL2, IL-6, CXCL10, FLT3L, VEGF, TGF-alpha IL-1RA, PD-L1, CX3CL1, TGF-beta1) were higher in MIS-C with severe myocarditis (MIS-C_MYO (CoV-2⁺)) (data not shown). Of note, 8 of them are known to be associated with TNF-α signaling. They are involved in propagation of inflammation (IL-6, IL-15), angiogenesis and vascular homeostasis (VEGF and TGF cytokines) and activation and chemotaxis of myeloid cells (CCL2, CX3CL1, CXCL10) (data not shown) (Holbrook et al., 2019; Varfolomeev and Ashkenazi, 2004). An increased level of CCL19, CCL20, CCL3 (cell migration and chemotaxis) and IL-1 agonist/antagonist (IL-1β, IL-1RA) were observed, as well as increased soluble PD-L1 (data not shown). Another noticeably elevated cytokine was CSF2, known to be involved in myeloid cell differentiation and migration (data not shown).

Altogether, high inflammatory cytokine levels were detected in both acute infection and postacute inflammatory cases. The strongest inflammatory profile was observed in MIS-C with severe myocarditis (MIS-C_MYO (CoV2⁺)) and remarkably, a similar profile was observed when comparing MIS-C without myocarditis (MIS-C (CoV2⁺)) and KD-like illness unrelated to SARS-CoV-2 (KD (CoV2⁻)). Of note, the inflammatory profile was much reduced in intensity in MIS-C cases with myocarditis under combined glucocorticosteroid and IVIG treatment, as compared to IVIG alone (data not shown).

Low Monocyte and Dendritic Cell Counts in Patients with Postacute Hyperinflammatory Illness

In each group (data not shown), PBMCs were analyzed by CyTOF mass spectrometry in combination with single-cell analyses at the transcriptomic level (SC-RNA-SEQ). Clustering analyses of the data obtained from CyTOF and SC-RNA-SEQ, revealed consistent results with most of the changes observed in clusters composed of monocytes or dendritic cells (data not shown). The most drastic changes were a decrease in conventional Dendritic Cells (cDCs) and plasmacytoid Dendritic Cells (pDCs) in patients with a postacute hyperinflammatory illness ((MIS-C_MYO (CoV2⁺), MIS-C (CoV2⁺) and KD (CoV2⁻)). As previously reported (Gruber et al., 2020), we also observed a trend towards a decrease in monocyte clusters in children with postacute hyperinflammatory illness, that was found independent of SARS-CoV-2 status (data not shown). Of note, some heterogeneity was observed in the proportions of non-classical monocyte in Acute-Inf (CoV2⁺). We observed additional heterogeneity in the proportions of classical and intermediate monocytes in patients with severe myocarditis (MIS-C_MYO (CoV2⁺) (data not shown), but there was no correlation with clinical data (data not shown), nor cytokine/chemokine measurements (data not shown). Additional modifications were detected in patients with acute SARS-CoV-2 infection (Acute-inf (CoV2⁺) cases), consisting in a decrease of MAIT cells and an excess of naïve and central memory CD4⁺ T cells (data not shown).

### Overexpression of Inflammatory Pathways, NF-κB Signaling and Metabolic Changes Related to Hypoxia in Acute Infection and Postacute Hyperinflammatory Conditions.

In patients with acute infection (Acute-inf (CoV2⁺) and Acute-inf (CoV2⁻)), the numbers of genes differentially expressed, in SC-RNA-SEQ analyses, were equally distributed among monocytes/DCs, T and B cells (data not shown). According to pathway enrichment analyses (Ingenuity Pathway Analyses; IPA, QIAGEN Inc. (Chen et al., 2013; Kuleshov et al., 2016), a decrease in oxidative phosphorylation, coupled with an increase of HMGB1 signaling, HIF-la signaling, hypoxia signaling, and production of nitric oxide was observed in both groups of acute infections, independently of SARS-CoV-2 infection, as compared to healthy controls (data not shown). These observations suggest a switch in metabolism potentially driven by hypoxic conditions. NF-κB signaling, VEGF signaling and inflammatory pathways (type-I and type II IFNs, IL-1, IL-6, IL-17) were also found to be overrepresented in both groups of patients (data not shown).

Interestingly, alterations in the very same pathways, were also identified in all cases of children with SARS-CoV-2-related postacute illnesses (All MIS-C (CoV2⁺): MIS-C_MYO (CoV2⁺) and MIS-C (CoV2⁺)). However, in these cases, alterations were mostly restricted to monocytes and dendritic cells (data not shown). Comparisons of genes differentially expressed between children with postacute hyperinflammatory illness with or without evidence of SARS-CoV-2 infection (All MIS-C (CoV2⁺) versus KD (CoV2⁻)), did not reveal significant differences with the exception of type-I and type-II interferon signaling (data not shown).

The NF-κB signaling pathway was identified to be activated in monocytes and DCs of all patients with acute infection and postacute hyperinflammatory illness, independently of SARS-CoV-2 infection (data not shown). While monocytes and DCs of patients with acute infection (Acute-inf (CoV2⁺) and Acute-inf (CoV2⁻)) highly expressed genes of the NF-κB complex (REL, RELA, RELB, NFKB1, NFKB2; data not shown), monocytes and DCs from all MIS-C patients (MIS-C_MYO (CoV2⁺) and MIS-C (CoV2⁺)) exhibited a strong decrease in the expression of NF-κB inhibitors, such as A20 (TNFAIP3), TNFAIP2, NFKBIA, NFKBID, NFKBIE and NFKBIZ (data not shown). Strikingly, this decrease in the expression of NF-κB inhibitors appeared to be quite specific to the monocytes and DCs of MIS-C patients with severe myocarditis (MIS-C_MYO (CoV2⁺)) (data not shown).

In conclusion, pathways dysregulated in acute infection or postacute hyperinflammatory illness, reflected an inflammatory status based on NF-κB signaling combined with changes in metabolism driven by a hypoxic environment. Whereas in acute respiratory disease gene expression changes likely reflected involvement of all PBMCs, monocytes and DCs were mostly affected in postacute hyperinflammatory illness.

A Gene Expression Signature Specific to MIS-C with Severe Myocarditis

To further gain insight into the inflammatory phenotype of monocytes and DCs from MIS-C patients, we compared single-cell gene expression between MIS-C patients with or without severe myocarditis and healthy controls (data not shown). Gene expression patterns highly differed among both groups of MIS-C, in particular in monocytes and DCs with a significant number of genes differentially expressed (data not shown). Type-I and type-II Interferon signaling pathways and several interferon stimulated genes (ISGs) (JAK2, STAT1, STAT2, IFITM1, IFITM2, IFI35, IFIT1, IFIT3, MX1, IRF1) were found to be only upregulated in the monocytes and DCs of MIS-C patients without myocarditis (data not shown), despite the fact that both groups of MIS-C patients showed elevated plasma IFN-α2 and IFNγ proteins (data not shown). Gene expression downregulation in monocytes and DCs of MIS-C patients with severe myocarditis, included most of the MEW class II genes suggesting a decrease in antigen processing and presentation pathways (data not shown). Following EnrichR pathways analysis, overexpression of targets of transcription factors belonging to the NF-κB complex were found highly enriched among genes upregulated in MIS-C patients with severe myocarditis by comparison with MIS-C without myocarditis (data not shown). This activation of NF-κB signaling in MIS-C patients with myocarditis was found to be correlated with the strong downregulation of NF-κB inhibitors. A strong overexpression of genes belonging to TNF-α signaling, as well as inflammatory responses, hypoxia and response to oxidative stress (HIF1A, HMOX1, HMBG1, . . . ) was found in cases with severe myocarditis (data not shown). This was associated with a decrease in expression of genes associated with oxidative phosphorylation, nitric oxide production and iNOS signaling (data not shown). TGF-β signaling and VEGF signaling were also found enriched in monocytes and DCs of patients with myocarditis and to a lesser magnitude in B cells (data not shown). Interestingly, an increased expression of several genes encoding S100 proteins and calcium-binding cytosolic proteins, all known to serve as danger signals to regulate cell migration, homeostasis and inflammation, were noticed in the cases of severe myocarditis (data not shown) (Xia et al., 2018).

To summarize, NF-κB activation, a decreased expression of NF-κB inhibitors, TNF-α signaling, together with an hypoxic response to oxidative stress, VEGF signaling, downregulation of MHC-II genes and a low type-I and type-II IFN responses characterize the monocytes and DCs of children with MIS-C and severe myocarditis.

Validation of a Molecular Signature Specific to MIS-C with Severe Myocarditis

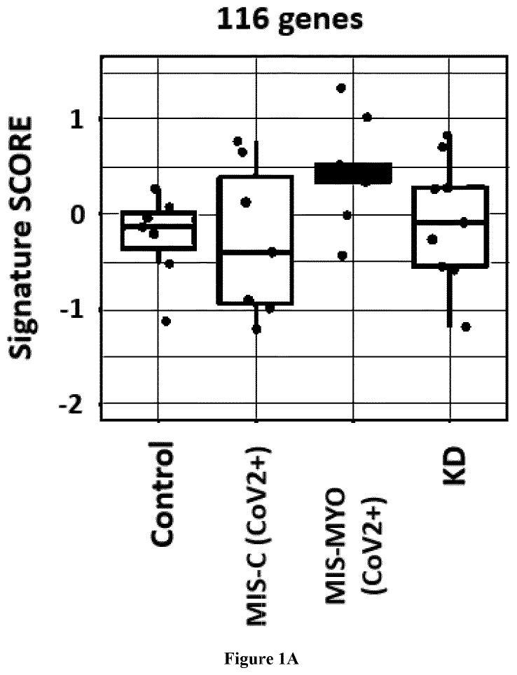

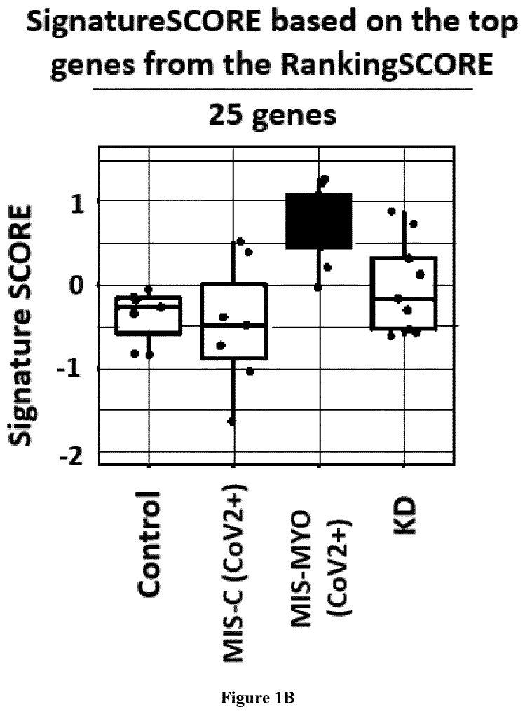

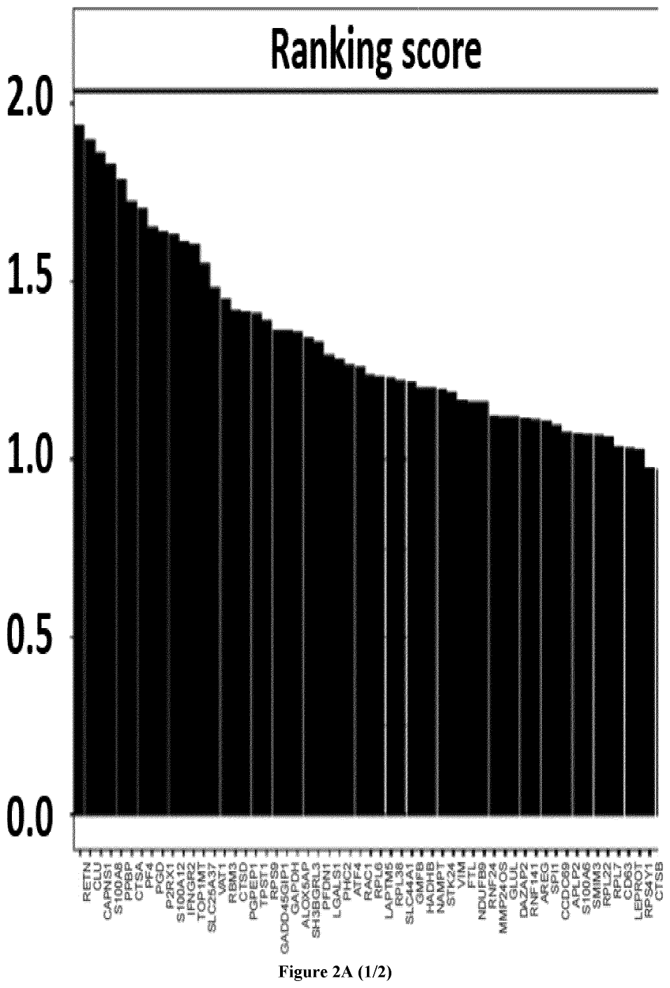

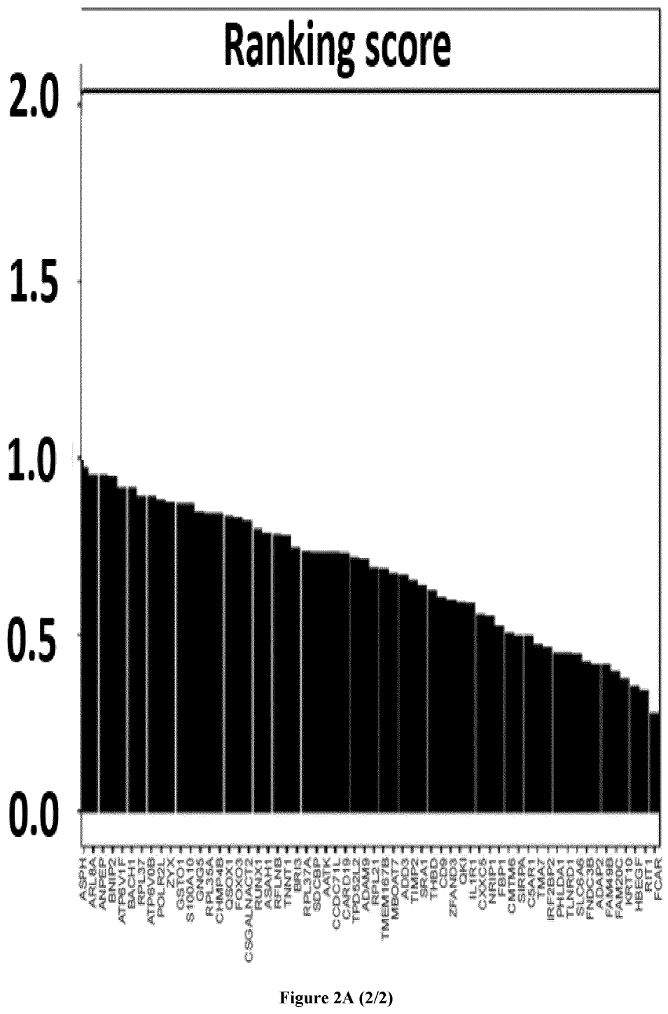

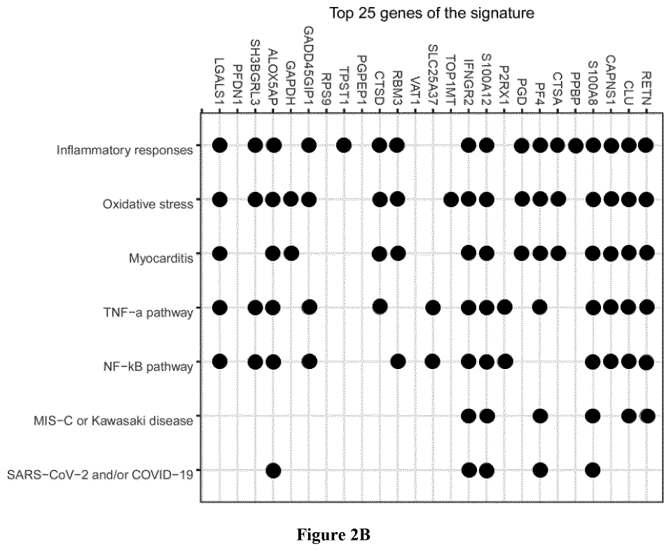

To identify a potential clinical relevance of our study, we searched for a molecular signature that correlated with the appearance of severe myocarditis among the monocytes/DCs of children with SARS-CoV-2-related MIS-C. By using several SC-RNA-SEQ comparison strategies (data not shown), we identified 329 genes upregulated in monocytes and DCs of the MIS-C group (N=6) with myocarditis as compared to all other groups (data not shown). To validate this molecular signature, RNA from PBMCs were sequenced from an independent group of patients. A scoring system was generated, based on normalized expression represented by a Z-score, coupled with hierarchical clustering, in order to identify genes that were overexpressed in children with myocarditis (MIS-C_MYO (CoV2⁺) group) as compared to the other groups (see Methods). Within the 329 genes identified by SC-RNA-SEQ in monocytes and DCs of patients with severe myocarditis, expression of 116 genes were found upregulated in PBMCs from an independent group of 9 patients belonging to the MIS-C_MYO (CoV2⁺) group with myocarditis (data not shown). From these genes, a signature score (SignatureSCORE) was determined for each sample processed by bulk-RNA-SEQ (FIG. 1A). We then further developed a RankingSCORE (FIG. 2A), to identify the top genes most contributing to the monocytes and DCs myocarditis signature. This led to the identification of a set of 25 genes, as the basis of a signatureSCORE that clearly segregate patients with severe myocarditis from other MIS-C and KD (CoV2⁻) (FIG. 1B). Consistently, most of these 25 genes belong to functional pathways that were previously identified (data not shown), such as inflammation, oxidative stress, TNF-α and/or NF-κB signaling, and in some cases already known markers of myocarditis or MIS-C and/or COVID-19, such as genes coding for S100 proteins (FIG. 2B).

### Test of the 116 Gene Signature on Myocarditis Post SARS-CoV-2 and Myocarditis Post SARS-CoV-2 Vaccination in Children and Adults

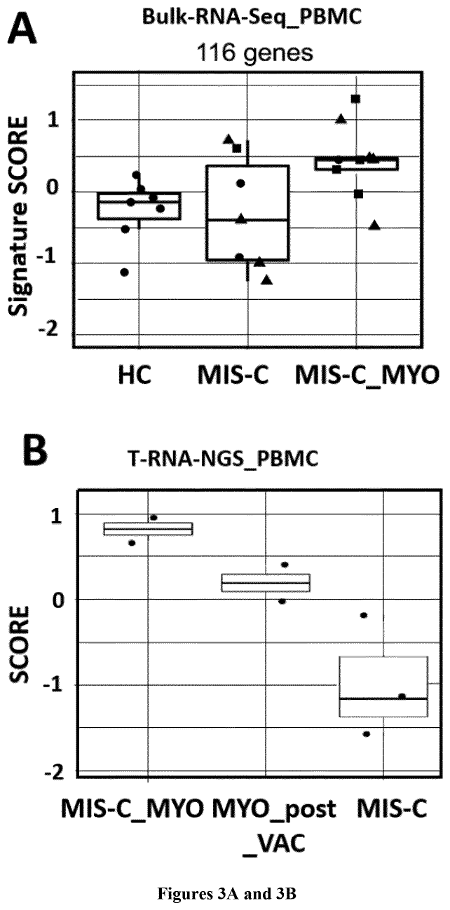

The signature of the 116 myocarditis genes (FIG. 3A) has been validated in children with MIS-C and severe myocarditis using collaborative T-RNA-NGS technology with Agilent (FIG. 3B). We have results also suggesting that two children who developed severe post-vaccination myocarditis have a high signature of these 116 genes (FIG. 3B).

### Test of the 116 Gene Signature Severe and Critical COVID-19 in Children and Adults

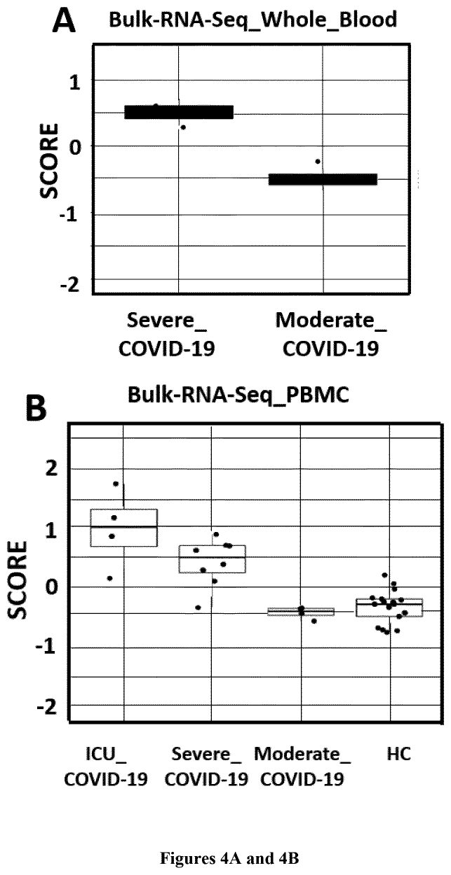

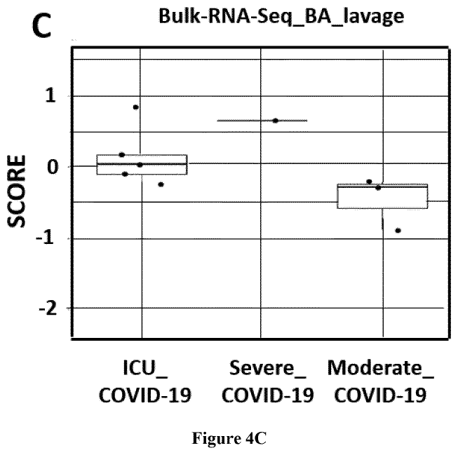

We also observed using this same technology (T-RNA-NGS) (preliminary results which will be extended to more samples later) (FIG. 4A) and based on data published in the literature (FIGS. 4B and 4C), that this signature of 116 genes also seemed to correlate with the degree of severity of COVID-19 pathology in adults.

### Discussion:

Multi-parametric analysis of peripheral blood mononuclear cells from children with acute respiratory infection and postacute hyperinflammation, detected an inflammatory profile associated with a loss of circulating monocytes and dendritic cells (DCs), as well as an upregulation of genes and pathways involving NF-kB signaling, oxidative stress with establishment of hypoxic conditions, actin cytoskeleton and VEGF signaling. These pathways were upregulated in both acute and postacute groups of patients, independently of SARS-CoV-2 infection. However, significant features of MIS-C with severe myocarditis were detected specifically in monocytes and DCs with low type-I and type-II IFNs responses, decrease of expression of NF-kB inhibitors, increased TNF-α signaling and overexpression of HIF-1α. Acute cases were characterized by the detection of inflammatory markers in the plasma with a particularly strong elevation of IL-8 and CXCL1, two chemokines known to mediate neutrophil migration to the lung (Kunkel et al., 1991; Pease and Sabroe, 2002; Sawant et al., 2015) and a modest elevation of IFNα2 levels. These findings suggest that in some children, a suboptimal anti-viral type-I interferon response, alongside a hyperinflammatory response (IL-6 levels and exacerbation of the NF-kB pathway), could account for SARS-CoV-2 disease with pneumonia, as compared to the very usual benign clinical course of SARS-CoV-2 infection in children. This has been previously observed in severe Respiratory Syncytial Virus (RSV) infections (Hijano et al., 2019).

In the postacute patients, elevated levels of plasma IFN-γ, IFNα2, IL-10, IL-15, and, to a lesser extent, TNF-α, were found, as previously described in other cohorts (Brodsky et al., 2020; Consiglio et al., 2020; Gruber et al., 2020). These findings are typical of an ongoing anti-viral immune response, not directly related to SARS-CoV-2 infection. In addition, elevated chemokines such as CCL2, CCL3 and CCL4 related to monocytes chemotaxis may recruit monocytes and DCs to tissues, possibly accounting for their reduced numbers observed in the blood of those patients. Additional mechanisms such as apoptosis or other cell death pathways may also be involved.

Cellular phenotypes that distinguish MIS-C from classical KD have been previously reported (Brodsky et al., 2020; Consiglio et al., 2020). Brodin and colleagues described several key differences such as elevated IL-17, IL-6 and CXCL10 that were only observed in KD, associated with decreased naïve CD4⁺ T cells and increased central memory and effector memory CD4⁺ T cells in MIS-C. In the present study, high (or even higher levels) of IL-17, IL-6 and CXCL10, were found in MIS-C cases as compared to the KD (CoV2⁻) group. In addition, no major differences in CD4 T cell compartments was detected. Accordingly, only a few differentially expressed genes were found between the MIS-C and KD (CoV2⁻) groups. These data support the hypothesis that MIS-C patients with KD features exhibit a molecular phenotype close to the one seen in KD patients, suggesting overlapping pathogenesis mechanisms. Differences observed with previous reports by Brodin and colleagues, may be due to inclusion of only patients with complete or incomplete KD criteria among the MIS-C cases, or technical differences in the respective studies, such as time of blood sampling relative to admission to hospital and medical treatments.

However, we did find noticeable differences in cases with severe myocarditis that required intensive care treatment. The expression of a number of cytokines was further increased in MIS-C with myocarditis, most of them related to the NF-κB-TNF-α signaling axis. Elevated VEGF and TGF-α and TGF-β are potential drivers of angiogenesis and vascular homeostasis, whereas elevated chemokines (CCL2, CCL3, CCL20, CX3CL1, CXCL10) could mediate increased cell migration towards inflamed tissues. Molecular analysis confirmed an upregulation of genes belonging to the TNF-α and NF-κB signaling pathways that were specifically found in monocytes and DCs of MIS-C patients with myocarditis. A lower expression of NF-κB complex inhibitors, including TNFAIP3 (A20), TNFAIP2, NFKBIA, NFKBIZ, was detected suggesting a possible mechanism for NF-κB sustained activation which could then potentially lead to exacerbated TNF-α signaling. Overall, these results point to a potential role of monocytes and DCs in the pathogenesis of MIS-C with severe myocarditis.

The apparent hypoxic conditions detected in children with myocarditis, could also account for the exacerbation of NF-κB signaling. HIF-1α, a sensor of oxidative stress, is well-known for being able to induce a switch from oxidative phosphorylation to glycolysis to limit generation of reactive oxygen species (ROS). It can also activate NF-κB signaling (D'Ignazio and Rocha, 2016; D'Ignazio et al., 2016). Additional environmental factors and/or genetic predispositions could also be involved. Another striking feature was the low-level expression of genes involved in type-I and type-II interferon responses, specifically in monocytes and DCs of children with myocarditis, although IFN-γ and IFNα2 proteins were elevated in the plasma of all MIS-C patients. This reduced response to type-I IFN in the most severe forms of MIS-C (with myocarditis) is in part reminiscent of the impaired type-I IFN activity observed in the most severe forms of COVID-19 in adults (Bastard et al., 2020; Hadjadj et al., 2020; Zhang et al., 2020). The search for auto-antibodies against IFNα2 were negative (data not shown) but presence of autoantibodies to ISG (interferon stimulated genes) cannot be excluded (Combes et al., 2021).

Overall, our findings depict a model, supported by previous publications (Amoah et al., 2015; Calabrese et al., 2004; Mann Douglas L., 2001), in which myocarditis is associated with an attenuated negative feedback loop of TNF-α-driven NF-κB activation, together with an excess of proangiogenic cytokines and chemokines that could attract activated myeloid and T cells to the myocardium tissue (not shown). Locally, it could lead to the production of inflammatory cytokines known to promote differentiation of cardiac fibroblasts into cardiac myofibroblasts (TNF-α, TGF-β, IL1-β, IL-13, IL-4, VEGF). Cardiac myofibroblasts, as previously reported, may secrete chemokines leading to further activation and recruitment of myeloid cells, creating a feed-forward loop of locally sustained inflammation and myocarditis (not shown) (Amoah et al., 2015; Angelo and Kurzrock, 2007; Delprat et al., 2020; Hua Xiumeng et al., 2020; Maloney and Gao, 2015).

Using SC-RNA-SEQ data, we defined a gene signature specific of SARS-CoV-2-related postacute hyperinflammatory illness with myocarditis that was further validated by a global transcriptomic analysis on PBMCs from an independent patient group. The genes defining this signature were consistently enriched in genes associated with inflammation, TNF-α and NF-κB signaling, oxidative stress and myocarditis (FIG. 2B). Interestingly, among these genes, the S100 proteins and the calprotectin complex (S100A8/S100A9) in particular, were previously reported and proposed as biomarkers for the most severe adult form of COVID-19 with acute respiratory syndrome (data not shown) (Silvin et al., 2020). Said signature would thus be suitable for predicting whether subjects suffering from a SARV-CoV-2 infection are at risk of having a multisystem inflammatory syndrome (MIS-C) with severe myocarditis or a disease severity following SARS-CoV-2 infection or myocarditis post-vaccination against SARS-CoV-2.

### REFERENCES

Throughout this application, various references describe the state of the art to which this invention pertains. The disclosures of these references are hereby incorporated by reference into the present disclosure.
Abrams, J. Y., Godfred-Cato, S. E., Oster, M. E., Chow, E. J., Koumans, E. H., Bryant, B., Leung, J. W., and Belay, E. D. (2020). Multisystem Inflammatory Syndrome in Children Associated with Severe Acute Respiratory Syndrome Coronavirus 2: A Systematic Review. J Pediatr 226, 45-54.e1.
Amoah, B. P., Yang, H., Zhang, P., Su, Z., and Xu, H. (2015). Immunopathogenesis of Myocarditis: The Interplay Between Cardiac Fibroblast Cells, Dendritic Cells, Macrophages and CD4+T Cells. Scandinavian Journal of Immunology 82, 1-9.
Angelo, L. S., and Kurzrock, R. (2007). Vascular Endothelial Growth Factor and Its Relationship to Inflammatory Mediators. Clin Cancer Res 13, 2825-2830.
Bagwell, C. B., Inokuma, M., Hunsberger, B., Herbert, D., Bray, C., Hill, B., Stelzer, G., Li, S., Kollipara, A., Ornatsky, O., et al. (2020). Automated Data Cleanup for Mass Cytometry. Cytometry A 97, 184-198.
Bastard, P., Rosen, L. B., Zhang, Q., Michailidis, E., Hoffmann, H.-H., Zhang, Y., Dorgham, K., Philippot, Q., Rosain, J., Béziat, V., et al. (2020). Autoantibodies against type I IFNs in patients with life-threatening COVID-19. Science 370.
Brodin, P. (2020). Why is COVID-19 so mild in children? Acta Paediatr 109, 1082-1083.
Brodsky, N. N., Ramaswamy, A., and Lucas, C. L. (2020). The Mystery of MIS-C Post-SARS-CoV-2 Infection. Trends in Microbiology 28, 956-958.
Calabrese, F., Carturan, E., Chimenti, C., Pieroni, M., Agostini, C., Angelini, A., Crosato, M., Valente, M., Boffa, G. M., Frustaci, A., et al. (2004). Overexpression of tumor necrosis factor (TNF) α and TNF α receptor I in human viral myocarditis: clinicopathologic correlations. Modern Pathology 17, 1108-1118.
Carter, M. J., Fish, M., Jennings, A., Doores, K. J., Wellman, P., Seow, J., Acors, S., Graham, C., Timms, E., Kenny, J., et al. (2020). Peripheral immunophenotypes in children with multisystem inflammatory syndrome associated with SARS-CoV-2 infection. Nature Medicine 26, 1701-1707.
Castagnoli, R., Votto, M., Licari, A., Brambilla, I., Bruno, R., Perlini, S., Rovida, F., Baldanti, F., and Marseglia, G. L. (2020). Severe Acute Respiratory Syndrome Coronavirus 2 (SARS-CoV-2) Infection in Children and Adolescents: A Systematic Review. JAMA Pediatr 174, 882-889.
CDC (2020). Multisystem Inflammatory Syndrome in Children (MIS-C) Associated with Coronavirus Disease 2019 (COVID-19).
Chang, L.-Y., Lu, C.-Y., Shao, P.-L., Lee, P.-I., Lin, M.-T., Fan, T.-Y., Cheng, A.-L., Lee, W.-L., Hu, J.-J., Yeh, S.-J., et al. (2014). Viral infections associated with Kawasaki disease. J Formos Med Assoc 113, 148-154.
Chen, E. Y., Tan, C. M., Kou, Y., Duan, Q., Wang, Z., Meirelles, G. V., Clark, N. R., and Ma'ayan, A. (2013). Enrichr: interactive and collaborative HTML5 gene list enrichment analysis tool. BMC Bioinformatics 14, 128.
Cheng, M. H., Zhang, S., Porritt, R. A., Rivas, M. N., Paschold, L., Willscher, E., Binder, M., Arditi, M., and Bahar, I. (2020). Superantigenic character of an insert unique to SARS-CoV-2 spike supported by skewed TCR repertoire in patients with hyperinflammation. PNAS 117, 25254-25262.
Combes, A. J., Courau, T., Kuhn, N. F., Hu, K. H., Ray, A., Chen, W. S., Chew, N. W., Cleary, S. J., Kushnoor, D., Reeder, G. C., et al. (2021). Global absence and targeting of protective immune states in severe COVID-19. Nature 1-10.
Consiglio, C. R., Cotugno, N., Sardh, F., Pou, C., Amodio, D., Rodriguez, L., Tan, Z., Zicari, S., Ruggiero, A., Pascucci, G. R., et al. (2020). The Immunology of Multisystem Inflammatory Syndrome in Children with COVID-19. Cell 183, 968-981.e7.
Darnell, M. E. R., and Taylor, D. R. (2006). Evaluation of inactivation methods for severe acute respiratory syndrome coronavirus in noncellular blood products. Transfusion 46, 1770-1777.
Datta, S. D., Talwar, A., and Lee, J. T. (2020). A Proposed Framework and Timeline of the Spectrum of Disease Due to SARS-CoV-2 Infection: Illness Beyond Acute Infection and Public Health Implications. JAMA 324, 2251-2252.
Delprat, V., Tellier, C., Demazy, C., Raes, M., Feron, O., and Michiels, C. (2020). Cycling hypoxia promotes a pro-inflammatory phenotype in macrophages via JNK/p65 signaling pathway. Scientific Reports 10, 882.
D'Ignazio, L., and Rocha, S. (2016). Hypoxia Induced NF-κB. Cells 5.
D'Ignazio, L., Bandarra, D., and Rocha, S. (2016). NF-κB and HIF crosstalk in immune responses. FEBS J 283, 413-424.
Gruber, C. N., Patel, R. S., Trachtman, R., Lepow, L., Amanat, F., Krammer, F., Wilson, K. M., Onel, K., Geanon, D., Tuballes, K., et al. (2020). Mapping Systemic Inflammation and Antibody Responses in Multisystem Inflammatory Syndrome in Children (MIS-C). Cell 183, 982-995.e14.
Grzelak, L., Temmam, S., Planchais, C., Demeret, C., Tondeur, L., Huon, C., Guivel-Benhassine, F., Staropoli, I., Chazal, M., Dufloo, J., et al. (2020). A comparison of four serological assays for detecting anti-SARS-CoV-2 antibodies in human serum samples from different populations. Sci Transl Med 12.
Gudbjartsson, D. F., Helgason, A., Jonsson, H., Magnusson, O. T., Melsted, P., Norddahl, G. L., Saemundsdottir, J., Sigurdsson, A., Sulem, P., Agustsdottir, A. B., et al. (2020). Spread of SARS-CoV-2 in the Icelandic Population. N Engl J Med 382, 2302-2315.
Hadjadj, J., Yatim, N., Barnabei, L., Corneau, A., Boussier, J., Smith, N., Pere, H., Charbit, B., Bondet, V., Chenevier-Gobeaux, C., et al. (2020). Impaired type I interferon activity and inflammatory responses in severe COVID-19 patients. Science 369, 718-724.
Han, H., Cho, J.-W., Lee, S., Yun, A., Kim, H., Bae, D., Yang, S., Kim, C. Y., Lee, M., Kim, E., et al. (2018). TRRUST v2: an expanded reference database of human and mouse transcriptional regulatory interactions. Nucleic Acids Res 46, D380-D386.
Hijano, D. R., Vu, L. D., Kauvar, L. M., Tripp, R. A., Polack, F. P., and Cormier, S. A. (2019). Role of Type I Interferon (IFN) in the Respiratory Syncytial Virus (RSV) Immune Response and Disease Severity. Front Immunol 10.
Holbrook, J., Lara-Reyna, S., Jarosz-Griffiths, H., and McDermott, M. F. (2019). Tumour necrosis factor signalling in health and disease. F1000Res 8.
Hua Xiumeng, Hu Gang, Hu Qingtao, Chang Yuan, Hu Yiqing, Gao Linlin, Chen Xiao, Yang Ping-Chang, Zhang Yu, Li Mingyao, et al. (2020). Single-Cell RNA Sequencing to Dissect the Immunological Network of Autoimmune Myocarditis. Circulation 142, 384-400.
Jones, V. G., Mills, M., Suarez, D., Hogan, C. A., Yeh, D., Segal, J. B., Nguyen, E. L., Barsh, G. R., Maskatia, S., and Mathew, R. (2020). COVID-19 and Kawasaki Disease: Novel Virus and Novel Case. Hosp Pediatr 10, 537-540.
Kanegaye, J. T., Wilder, M. S., Molkara, D., Frazer, J. R., Pancheri, J., Tremoulet, A. H., Watson, V. E., Best, B. M., and Burns, J. C. (2009). Recognition of a Kawasaki disease shock syndrome. Pediatrics 123, e783-789.
Kim, D., Paggi, J. M., Park, C., Bennett, C., and Salzberg, S. L. (2019). Graph-based genome alignment and genotyping with HISAT2 and HISAT-genotype. Nat Biotechnol 37, 907-915.
Kuleshov, M. V., Jones, M. R., Rouillard, A. D., Fernandez, N. F., Duan, Q., Wang, Z., Koplev, S., Jenkins, S. L., Jagodnik, K. M., Lachmann, A., et al. (2016). Enrichr: a comprehensive gene set enrichment analysis web server 2016 update. Nucleic Acids Res 44, W90-97.
Kunkel, S. L., Standiford, T., Kasahara, K., and Strieter, R. M. (1991). Interleukin-8 (IL-8): the major neutrophil chemotactic factor in the lung. Exp Lung Res 17, 17-23.
Levy, C., Basmaci, R., Bensaid, P., Bru, C. B., Coinde, E., Dessioux, E., Fournial, C., Gashignard, J., Haas, H., Hentgen, V., et al. (2020). Changes in RT-PCR-positive SARS-CoV-2 rates in adults and children according to the epidemic stages. MedRxiv 2020.05.18.20098863.
Love, M. I., Huber, W., and Anders, S. (2014). Moderated estimation of fold change and dispersion for RNA-seq data with DESeq2. Genome Biol 15, 550.
Maloney, J. P., and Gao, L. (2015). Proinflammatory Cytokines Increase Vascular Endothelial Growth Factor Expression in Alveolar Epithelial Cells (Hindawi).
Mann Douglas L. (2001). Tumor Necrosis Factor and Viral Myocarditis: The Fine Line Between Innate and Inappropriate Immune Responses in the Heart. Circulation 103, 626-629.
McCrindle, B. W., Rowley, A. H., Newburger, J. W., Burns, J. C., Bolger, A. F., Gewitz, M., Baker, A. L., Jackson, M. A., Takahashi, M., Shah, P. B., et al. (2017). Diagnosis, Treatment, and Long-Term Management of Kawasaki Disease: A Scientific Statement for Health Professionals From the American Heart Association. Circulation 135, e927-e999.
McInnes, L., Healy, J., and Melville, J. UMAP: Uniform Manifold Approximation and Projection for Dimension Reduction. 63.
Monaco, G., Lee, B., Xu, W., Mustafah, S., Hwang, Y. Y., Cane, C., Burdin, N., Visan, L., Ceccarelli, M., Poidinger, M., et al. (2019). RNA-Seq Signatures Normalized by mRNA Abundance Allow Absolute Deconvolution of Human Immune Cell Types. Cell Rep 26, 1627-1640.e7.
Pease, J. E., and Sabroe, I. (2002). The Role of Interleukin-8 and its Receptors in Inflammatory Lung Disease. Am J Respir Med 1, 19-25.
Rodero, M. P., Decalf, J., Bondet, V., Hunt, D., Rice, G. I., Werneke, S., McGlasson, S. L., Alyanakian, M.-A., Bader-Meunier, B., Barnerias, C., et al. (2017). Detection of interferon alpha protein reveals differential levels and cellular sources in disease. J Exp Med 214, 1547-1555.
Sawant, K. V., Xu, R., Cox, R., Hawkins, H., Sbrana, E., Kolli, D., Garofalo, R. P., and Rajarathnam, K. (2015). Chemokine CXCL1-Mediated Neutrophil Trafficking in the Lung: Role of CXCR2 Activation. J Innate Immun 7, 647-658.
Silvin, A., Chapuis, N., Dunsmore, G., Goubet, A.-G., Dubuisson, A., Derosa, L., Almire, C., Hénon, C., Kosmider, O., Droin, N., et al. (2020). Elevated Calprotectin and Abnormal Myeloid Cell Subsets Discriminate Severe from Mild COVID-19. Cell 182, 1401-1418.e18.
Stuart, T., Butler, A., Hoffman, P., Hafemeister, C., Papalexi, E., Mauck, W. M., Hao, Y., Stoeckius, M., Smibert, P., and Satija, R. (2019). Comprehensive Integration of Single-Cell Data. Cell 177, 1888-1902.e21.
Tagarro, A., Epalza, C., Santos, M., Sanz-Santaeufemia, F. J., Otheo, E., Moraleda, C., and Calvo, C. (2020). Screening and Severity of Coronavirus Disease 2019 (COVID-19) in Children in Madrid, Spain. JAMA Pediatr.
Toubiana, J., Poirault, C., Corsia, A., Bajolle, F., Fourgeaud, J., Angoulvant, F., Debray, A., Basmaci, R., Salvador, E., Biscardi, S., et al. (2020). Kawasaki-like multisystem inflammatory syndrome in children during the covid-19 pandemic in Paris, France: prospective observational study. BMJ 369, m2094.
Toubiana, J., Cohen, J. F., Brice, J., Poirault, C., Bajolle, F., Curtis, W., Moulin, F., Matczak, S., Leruez, M., Casanova, J.-L., et al. (2021). Distinctive Features of Kawasaki Disease Following SARS-CoV-2 Infection: a Controlled Study in Paris, France. J Clin Immunol.
Van Gassen, S., Callebaut, B., Van Helden, M. J., Lambrecht, B. N., Demeester, P., Dhaene, T., and Saeys, Y. (2015). FlowSOM: Using self-organizing maps for visualization and interpretation of cytometry data. Cytometry A 87, 636-645.
Varfolomeev, E. E., and Ashkenazi, A. (2004). Tumor Necrosis Factor: An Apoptosis JuNKie? Cell 116, 491-497.
Whittaker, E., Bamford, A., Kenny, J., Kaforou, M., Jones, C. E., Shah, P., Ramnarayan, P., Fraisse, A., Miller, O., Davies, P., et al. (2020). Clinical Characteristics of 58 Children With a Pediatric Inflammatory Multisystem Syndrome Temporally Associated With SARS-CoV-2. JAMA 324, 259-269.
Wiersinga, W. J., Rhodes, A., Cheng, A. C., Peacock, S. J., and Prescott, H. C. (2020). Pathophysiology, Transmission, Diagnosis, and Treatment of Coronavirus Disease 2019 (COVID-19): A Review. JAMA 324, 782-793.
Xia, C., Braunstein, Z., Toomey, A. C., Zhong, J., and Rao, X. (2018). S100 Proteins As an Important Regulator of Macrophage Inflammation. Front. Immunol. 8.
Zhang, Q., Bastard, P., Liu, Z., Pen, J. L., Moncada-Velez, M., Chen, J., Ogishi, M., Sabli, I. K. D., Hodeib, S., Korol, C., et al. (2020). Inborn errors of type I IFN immunity in patients with life-threatening COVID-19. Science 370.
Zimmermann, P., and Curtis, N. (2020). Coronavirus Infections in Children Including COVID-19: An Overview of the Epidemiology, Clinical Features, Diagnosis, Treatment and Prevention Options in Children. Pediatr Infect Dis J 39, 355-368.

1. A method of predicting whether
i) a subject suffering from a SARS-CoV-2 infection is at risk of having a multisystem inflammatory syndrome (MIS-C) with severe myocarditis; or
ii) a subject suffering from a SARS-CoV-2 infection is at risk of having a severe or critical form of C OVID-19; or
iii) a subject is a risk of having myocarditis post-vaccination against SARS-CoV-2; and treating the subject, comprising
determining an expression level in a sample obtained from the subject of at least one gene selected from the group consisting of
RETN
CLU
CAPNS1
S100A8
PPBP
CTSA
PF4
PGD
P2RX1
S100A12
IFNGR2
TOP1MT
SLC25A37
VAT1
RBM3
CTSD
PGPEP1
TPST1
RPS9
GADD45GIP1
GAPDH
ALOX5AP
SH3BGRL3
PFDN1
LGALS1
PHC2
ATF4
RAC1
RPL6
LAPTM5
RPL38
SLC44A1
GMFB
HADHB
NAMPT
STK24
VIM
FTL
NDUFB9
RNF24
MMP24OS
GLUL
DAZAP2
RNF141
AREG
SPI1
CCDC69
APLP2
S100A6
SMIM3
RPL22
RPL7
CD63
LEPROT
RPS4Y1
CTSB
ASPH
ARL8A
ANPEP
BNIP2
ATP6V1F
BACH1
RPL37
ATP6V0B
POLR2L
ZYX
GSTO1
S100A10
GNG5
RPL35A
CHMP4B
QSOX1
FOXO3
CSGALNACT2
RUNX1
ASAH1
RFLNB
TNNT1
BRI3
RPL37A
SDCBP
AATK
CCDC71L
CARD19
TPD52L2
ADAM9
RPL21
TMEM167B
MBOAT7
ADD3
TIMP2
SRA1
THBD
CD9
ZFAND3
QKI
IL1R1
CXXC5
NRIP1
FBP1
CMTM6
SIRPA
C5AR1
TMA7
RF2BP2
PHLDA1
TLNRD1
SLC6A6
FNDC3B
ADAP2
FAM49B
FAM20C
KRT10
HBEGF
RIT1
FCAR
administering one or more of a corticosteroid, an intravenous immunoglobulin (IVIG) and a TNF blocking agent to a subject identified as having an expression level of the at least one gene that is higher than a predetermined reference value.

2. (canceled)

3. (canceled)

4. The method of claim 1, wherein the subject is child or an adult.

5. The method of claim 1 wherein the expression levels of:
RETN
CLU
CAPNS1
S100A8
PPBP
CTSA
PF4
PGD
P2RX1
S100A12
IFNGR2
TOP1MT
SLC25A37
VAT1
RBM3
CTSD
PGPEP1
TPST1
RPS9
GADD45GIP1
GAPDH
ALOX5AP
SH3BGRL3
PFDN1
LGALS1
are determined and the sample is obtained from a child suffering from a SARS-CoV-2 infection.

6. The method of claim 1, wherein the sample is a whole blood sample, a PMBC sample or a sample of monocytes.

7. The method of claim 1, wherein the expression level of the at least one gene is determined by RNA sequencing.

8. The method of claim 1, wherein the higher is the expression level of the at least one gene, the higher is the risk of having MIS-C with severe myocarditis or a severe or critical form of COVID-19 or myocarditis post-vaccination against SARS-CoV-2.

9. The method of claim 1, comprising comparing the expression level with a predetermined reference value wherein detecting a difference between the expression level and the predetermined reference value indicates the risk of a MIS-C with severe myocarditis or a severe or critical form of COVID-19 or myocarditis post-vaccination against SARS-CoV-2.

10. The method of claim 9 wherein when the expression level is higher than the predetermined reference value, then it is concluded that the subject has a high risk of having a MIS-C with severe myocarditis or a severe or critical form of COVID-19 or myocarditis post-vaccination against SARS-CoV-2 whereas when the expression level is lower than the predetermined reference value, then it is concluded that the subject has a low risk of having a MIS-C with severe myocarditis or a severe or critical form of COVID-19 or myocarditis post-vaccination against SARS-CoV-2.

11. The method of claim 1, wherein a score is calculated.

12. The method of claim 1, comprising a) determining the expression level of the at least one gene in the sample obtained from the subject; b) implementing an algorithm on data comprising the expression level so as to obtain an algorithm output; c) determining the risk of having a MIS-C with severe myocarditis or a severe or critical form of COVID-19 or myocarditis post-vaccination against SARS-CoV-2 from the output obtained at step c).

13. (canceled)

14. The method of claim 1, wherein a corticosteroid in combination with IVIG is administered to the subject.

15. The method of claim 1, wherein the sample a sample containing immune cells.
